(19) United States
(12) Patent Application Publication
(10) Pub. No.: US 2010/0124759 A1
(43) Pub. Date: May 20, 2010

(54) MICROFLUIDIC DROPLETS FOR METABOLIC ENGINEERING AND OTHER APPLICATIONS

(75) Inventors: Benjamin L. Wang, Somerville, MA (US); Katherine J. Humphry, Cambridge, MA (US); David A. Weitz, Bolton, MA (US); Gregory Stephanopoulos, Winchester, MA (US)

Correspondence Address: WOLF GREENFIELD & SACKS, P.C., 600 ATLANTIC AVENUE, BOSTON, MA, 02210-2206, US

(73) Assignees: Massachusetts Institute of Technology, Cambridge, MA (US); President and Fellows of Harvard College, Cambridge, MA (US)

(21) Appl. No.: 12/492,830

(22) Filed: Jun. 26, 2009

Related U.S. Application Data
(60) Provisional application No. 61/076,473, filed on Jun. 27, 2008.

(51) Int. Cl. C12Q 1/28; C12N 1/18; C12Q 1/26; C12N 1/20

(52) U.S. Cl. 435/28; 435/255.2; 435/25; 435/252.8

(57) ABSTRACT

The present invention relates generally to the use of droplets to culture and/or assay cells or other species. In some cases, the cells or other species may be sorted based upon the results of the culture and/or assay. In some embodiments, cells other species can be encapsulated in droplets and exposed to one or more agents (e.g., a sugar, an indicator dye, etc.). For instance, in some cases, exposure of cells to the agents may result in the production of metabolites or other compounds (e.g., amino acids, proteins, organic acids, etc.) which may be, for example, assayed or otherwise determined. In some embodiments, the reaction of an agent with cells and/or other species within a droplet may reveal a property of the cells or other species (e.g., sugar consumption, growth rate, ability to withstand exposure to the agent, etc.). As an example, cells that produce desired metabolites or exhibit certain properties may be separated from the other cells via sorting techniques. Other aspects of the invention relate to devices or kits for implementing such sorts, methods of promoting such techniques, or the like.

### RELATED APPLICATIONS

This application claims the benefit of U.S. Provisional Patent Application Ser. No. 61/076,473, filed Jun. 27, 2008, entitled “Microfluidic Droplets for Metabolic Engineering and Other Applications,” by Wang, et al., the entirety of which is incorporated herein by reference.

### GOVERNMENT FUNDING

Research leading to various aspects of the present invention were sponsored, at least in part, by the National Science Foundation, Grant No. BES-0331364. The U.S. Government has certain rights in the invention.

### FIELD OF INVENTION

The present invention relates generally to the use of droplets to culture and/or assay cells or other species. In some cases, the cells or other species may be sorted based upon the results of the culture and/or assay.

### BACKGROUND

Metabolic engineering has contributed significantly to the improvement of genetic strains for industrial and other applications. For instance, genes useful in the production of product, or other so-called distal genes may be manipulated using various metabolic engineering techniques to impact the production of a product, for example, due to kinetic or regulatory effects. Such genes can be identified, in some cases, by combinatorial methods where libraries are constructed that contain one or more genes and/or random variants of such genes, random combinations of gene knock outs, over-expressions, or the like. Cells with superior properties can be selected from these libraries and the specific genetic alteration identified using processes such as inverse metabolic engineering. These approaches often benefit from the use of high throughput screening methods to select desirable clones from these libraries. For many libraries, selection criteria that can be used includes the production of a secreted metabolite or the consumption of a medium component. A strategy for compartmentalizing clones may be useful where each clone grows in a separate environment, which can allow for the measurement of clone-specific metabolite concentrations or the like.

Traditional methods that make use of such instruments as microwell plates for culturing and assaying can be utilized in various compartmentalizing strategies. However, such methods may not provide sufficiently high throughput. Accordingly, improved compositions and methods are needed.

### SUMMARY OF THE INVENTION

The present invention relates generally to the use of droplets to culture and/or assay cells or other species. In some cases, the cells or other species may be sorted based upon the results of the culture and/or assay. The subject matter of the present invention involves, in some cases, interrelated products, alternative solutions to a particular to problem, and/or a plurality of different uses of one or more systems and/or articles.

In one aspect, the method is a method of producing an enriched population of cells. In one set of embodiments, the method includes acts of providing a first population of droplets contained within a microfluidic device, at least some of which droplets encapsulate one or more cells, at least some of which droplets include a first cell type and at least some of which droplets include a second cell type; for at least some of the droplets, determining the ability of one or more cells within the respective droplets to react with a sugar, wherein the first cell type is able to metabolize the sugar to a greater degree than the second cell type; and based on the determination, producing an enriched population of droplets of cells of the first cell type relative to the second cell type.

In some embodiments, the method includes acts of providing a population of droplets contained within a microfluidic device, at least some of which droplets encapsulate one or more cells, at least some of which droplets of the population of droplets include a first cell type and at least some of which droplets include a second cell type; for at least some of the droplets, determining the ability of one or more cells within the droplet to react with an agent, wherein the first cell type reacts with the agent to a greater degree than does the second cell type; and based on the determination, producing an enriched population of droplets of cells of the first cell type relative to the second cell type.

The method, according to another aspect, is generally directed to a method of producing an enriched population of a species. In one set of embodiments, the method includes acts of providing a population of droplets contained within a microfluidic device, at least some of which droplets encapsulate a first species and at least some of which droplets of the population of droplets include a second species; for at least some of the droplets, determining the ability of one or more species within the droplet to react with an agent, wherein the first species reacts with the agent to a greater degree than does the second species; and based on the determination, producing an enriched population of droplets containing the first species relative to the droplets containing the second species.

In yet another aspect, the method includes acts of providing a population of droplets contained within a microfluidic device, at least some of which droplets encapsulate one or more cells and at least some of which droplets contain a sugar; exposing at least some of the droplets to an enzyme able to react with the sugar; and to determining an extent of reaction of the enzyme with the sugar.

The method, according to still another aspect, includes acts of exposing a population of droplets contained within a microfluidic device, at least some of which droplets encapsulate one or more cells, to a sugar for a period of time at least sufficient to allow the sugar to enter at least some of the droplets; exposing at least some of the droplets to an enzyme able to react with the sugar; and determining an extent of reaction of the enzyme with the sugar.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic diagram of a device according to one embodiment;

FIG. 2 is a schematic diagram of a device according to another embodiment;

FIG. 3A is a schematic diagram of a microfluidic high-throughput screening platform according to yet another embodiment;

FIG. 3B is a schematic diagram of a droplet making device according to still another embodiment;

FIG. 4 is an optical image of single cells encapsulated in droplets prior to culturing according to one embodiment;

FIG. 5 is an optical image of multiple cells encapsulated in droplets after culturing according to another embodiment;

FIG. 6 is a schematic diagram of a device used to perform assay reactions in droplets, according to yet another embodiment;

FIG. 7 is a schematic diagram of an integrated device including a section for the coalescence of droplets according to another embodiment;

FIG. 8 is a plot of fluorescence detection data from H131 strain after 2 days of culturing in another embodiment;

FIG. 9 is a plot of fluorescence detection data from H131 and TAL1 strains after 2 days of culturing, in still another embodiment;

FIG. 10 is a plot of the percentage of cells within a given fluorescence range for H131 and TAL1 strains after 2 days of culturing according to yet another embodiment;

FIG. 11 is a plot of the percentage of cells within a given fluorescence range for H131 and TAL1 strains after 3 days of culturing according to still another embodiment;

FIG. 12 is a plot of the percentage of cells within a given fluorescence range for H131 and TAL1 strains after 3 days of culturing according to another embodiment;

FIG. 13 is a schematic diagram of a device including sections for coalescence, detection and sorting according to still another embodiment;

FIG. 14 is a schematic illustration of a genomic DNA library construction, according to one set of embodiments;

FIG. 15 includes a schematic illustration of XYLA gene construct in H131-A31, according to one set of embodiments;

FIG. 16 is an exemplary plot illustrating the percentage of droplets in different fluorescence ranges;

FIG. 17 is an exemplary plot illustrating the percentage of droplets in different fluorescence ranges;

FIG. 18 includes plots of xylose consumption for rich and minimal media, according to one set of embodiments; and

FIGS. 19A-19D include plots of xylose consumption for various mutants, according to one set of embodiments.

### DETAILED DESCRIPTION

The present invention relates generally to the use of droplets to culture and/or assay cells or other species. In some cases, the cells or other species may be sorted based upon the results of the culture and/or assay. In some embodiments, cells or other species can be encapsulated in droplets and exposed to one or more agents (e.g., a sugar, an indicator dye, etc.). For instance, in some cases, exposure of cells to the agents may result in the production of metabolites or other compounds (e.g., amino acids, proteins, organic acids, etc.) which may be, for example, assayed or otherwise determined. In some embodiments, the reaction of an agent with cells and/or other species within a droplet may reveal a property of the cells or other species (e.g., sugar consumption, growth rate, ability to withstand exposure to the agent, etc.). As an example, cells that produce desired metabolites or exhibit certain properties may be separated from the other cells via sorting techniques. Other aspects of the invention relate to devices or kits for implementing such sorts, methods of promoting such techniques, or the like.

One aspect of the invention is generally directed to systems and methods of sorting cells, e.g., to produce an enriched population of cells. In some cases, a population of cells is contained within a plurality of droplets, and the droplets are sorted to produce an enriched population of cells. As discussed below, in some cases, the cells may be sorted based on their reaction with an agent delivered to the droplets containing the cells.

The methods and devices described herein can be used, in some embodiments, to produce an enriched population of cells or other species. For instance, a population of cells, including a first cell type and a second cell type, may be sorted to produce a population of cells enriched in the first cell type relative to the second cell type, i.e., such that the percentage of cells of the first cell type, expressed as a percentage of the overall number of cells, is higher after sorting than before sorting. The cells (or other species) may then be subsequently used in various applications. For instance, products may be harvested from the droplets, the droplets may be combined with other droplets, the species may be further purified, the cells may be cultured, or the like. As a specific example, in some embodiments, the DNA of an enriched population of cells may be sequenced, for example, to determine the presence and/or identity of a desirable or an undesirable gene. Various methods for sequencing the DNA of a cell are known to those of ordinary skill in the art, for example, PCR (polymerase chain reaction) techniques.

In various embodiments, the amount of enrichment of the cells or other species after sorting may be by a factor of at least about 3, at least about 5, at least about 10, at to least about 15, at least about 20, at least about 25, at least about 30, at least about 100, at least about 1000, or at least about 10,000 or more in some cases. In one set of embodiments, a population of cells (or other species) enriched in one cell type may be subjected to one or more additional rounds of screening, e.g., as discussed herein, which may result in even higher degrees of enrichment of a cell type relative to other cell types in the population.

Certain embodiments of the present invention are generally directed to the encapsulation of one or more cells in droplets. As used herein, a “cell” is given its ordinary meaning as used in biology. The cell may be any cell or cell type. For example, the cell may be a bacterium or other single-cell organism, a plant cell, or an animal cell. If the cell is a single-cell organism, then the cell may be, for example, a protozoan, a trypanosome, an amoeba, a yeast cell, algae, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell such as a primate cell, a bovine cell, a horse cell, a porcine cell, a goat cell, a dog cell, a cat cell, or a cell from a rodent such as a rat or a mouse. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, a osteocyte, a muscle cell, a blood cell, an endothelial cell, an immune cell (e.g., a T-cell, a B-cell, a macrophage, a neutrophil, a basophil, a mast cell, an eosinophil), a stem cell, etc. In some cases, the cell may be a genetically engineered cell. In certain embodiments, the cell may be a Chinese hamster ovarian (“CHO”) cell or a 3T3 cell.

It should be understood, however, that the present invention is not limited to only the culturing and/or sorting of cells contained within droplets, but is also applicable to the sorting of any other species that may be contained within droplets, for instance, biochemical species such as nucleic acids such as siRNA, RNAi and DNA, proteins, peptides, or enzymes. Additional species that can be incorporated within a droplet include, but are not limited to, nanoparticles, quantum dots, fragrances, proteins, indicators, dyes, fluorescent species, chemicals, or the like. For instance, a population of droplets containing a first type of quantum dot and a second type of quantum dot may be sorted, e.g., on the basis of fluorescence, as discussed herein. Accordingly, the use of cells contained within droplets as discussed herein is by way of example only.

In some embodiments, the cells or other species are sorted based on their ability to react with an agent, e.g., contained within the droplet. The agent may be delivered to the cell or other species within the droplet using any suitable technique, for instance, via diffusion from the carrier solution, via coalescence of the droplet containing the cell with another droplet containing the agent, or the like. Systems and methods for use in the coalescence of droplets are described in U.S. patent application Ser. No. 11/360,845, filed Feb. 23, 2006, entitled “Electronic Control of Fluidic Species,” published as U.S. Patent Application Publication No. 2007/000342 on Jan. 4, 2007, or in U.S. patent application Ser. No. 11/698,298, filed Jan. 24, 2007, entitled “Fluidic Droplet Coalescence,” each incorporated herein by reference. For instance, in some cases, the carrier phase may comprise one or more agents capable of reacting with the cells. In some cases, the population of droplets is exposed to an agent (e.g., a sugar) for a period of time at least sufficient to allow the agent to enter at least some of the droplets.

In some cases, more than one agent may be introduced into a droplet. For instance, a first agent may be introduced into the droplet and allowed to react with the cell or other species, then a second agent introduced into the droplet to determine the first agent, e.g., concentration or amount of the first agent present within the droplet after reaction. As specific examples, the agent may comprise a chemical capable of reacting with the cell, with a product produced by the cell, and/or with another species previously introduced to the cell and/or to the droplet containing the cell. As another example, the second agent may comprise an enzyme (e.g., an oxidase) able to react with a sugar (e.g., xylose) that was introduced into the droplet, e.g., during formation of the droplet. In some embodiments, the second agent may comprise a chemical capable of reacting with an entity produced by the cell before and/or after exposure to the original agent. The second agent may comprise, in some instances, a chemical capable of reacting with a cell only after the cell has reacted and/or not reacted with the original agent introduced during the droplet formation phase.

In some embodiments, the agent comprises a species that can be at least partially metabolized by a cell, e.g., to produce one or more metabolites from the cell. For example, the agent may comprise a sugar (e.g., xylose, deoxyribose, sucrose, fructose, glucose, galactose, etc.) or other suitable carbohydrate. As another example, in some to embodiments, the agent may comprise an amino acid (e.g., aspartic acid, lysine, etc.). The agent may also comprise, in some cases, nucleic acids such as RNA, siRNA, RNAi, DNA, PNA, etc. and/or other species such as proteins, peptides, enzymes, etc.

In one set embodiments, the droplet may contain a sugar and an oxidase able to oxidize that sugar. For instance, the oxidase may be a carbohydrate oxidase or an oligosaccharide oxidase, e.g., pyranose oxidase, galactose oxidase, glucose oxidase, etc. In some cases, the agent may also comprise an additional enzyme such as horseradish peroxidase. In some cases, the agent may also comprise an indicator dye, such as Amplex UltraRed (Molecular Probes), Amplex Red (Molecular Probes), dihydrofluoresceins, dihydrorhodamines, etc. For instance, in some cases, exposure of the sugar to the enzyme may cause the production of hydrogen peroxide. The hydrogen peroxide, in some cases, can then be assayed to determine the sugar. For instance, the hydrogen peroxide may be reacted with a non-fluorescent compound, such as Amplex UltraRed (Molecular Probes), to produce a fluorescent compound, such as Resorufin.

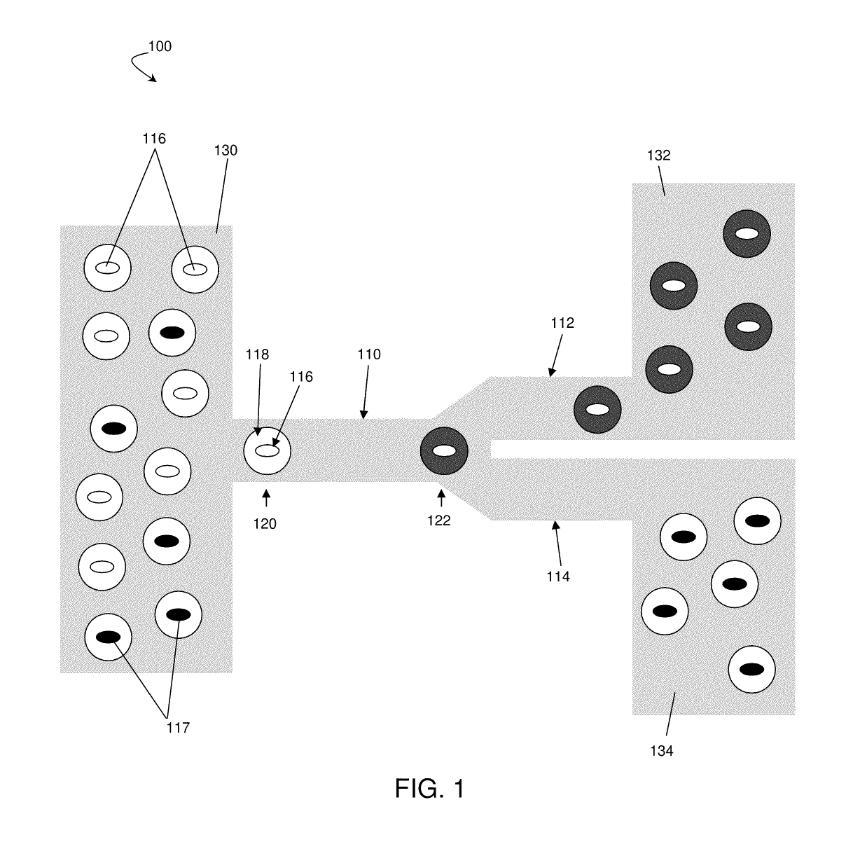

A non-limiting illustrative example of a system of the invention is discussed below with reference to FIG. 1. As shown in this example, device 100 includes an entry channel 110, and two exit channels 112 and 114. In some cases, device 100 is a microfluidic device, As described in more detail later, the channels of the device may be of any depth, width, and/or height, and the channels may each define any path (e.g., straight, meandering, etc.). In some cases (including the one illustrated in FIG. 1), a first type of cell 116 (or other species) is encapsulated in a droplet 118. In addition, a second type of cell 117 may also be encapsulated in a droplet as shown, for example, in optional chamber 130. In other cases, the droplet may contain more than one cell, or it may not contain any cells. Each droplet, in some embodiments, may contain exactly one cell type, while in other embodiments, more than one cell type may be contained within a single droplet.

In the example shown in FIG. 1, an agent is introduced into droplet 118 between points 120 and 122 in channel 110. The agent may be introduced into the droplet by any suitable technique, for instance, via diffusion, via the coalescence of droplet 118 with another droplet containing the agent, or the like. The cell may react with the agent in some cases, while in others, the cell may not react. For example, exposure of a cell to an agent may result in the death of the cell. Alternatively, the cell may survive exposure to the agent, and in some cases, the cell may be able to metabolize or otherwise utilize the agent. For instance, in some cases, the exposure of a cell to an agent may cause a change in the growth rate of the cell, a change in the production of one or more metabolites, or a change in another property of the cell (e.g., fluorescence, color, morphology, size, mitotic ability, etc.). In some instances, the agent may react with another species in the droplet (e.g., nucleic acids such as RNA or DNA, proteins or peptides, enzymes, antibodies, etc.) in addition to and/or instead of the cell. In some cases, the reaction of an agent with a cell and/or other species may result in a determinable change in a property of the droplet and/or its contents (e.g., a change in fluorescence, a change in color, etc.).

The extent of reaction between the agent and the cell and/or other species may be determined, for example, at some point within the microfluidic system. For instance, in the example illustrated in FIG. 1, the determining step can take place at point 122. Examples of characteristics determinable within the droplet and usable in the invention may be identified by those of ordinary skill in the art, and include, but are not limited to, fluorescence, spectroscopy (e.g., optical, infrared, ultraviolet, etc.), radioactivity, mass, volume, density, temperature, viscosity, pH, concentration of a substance, such as a biological substance (e.g., a protein, a nucleic acid, etc.), viability of one or more cells within the droplet, or the like. Those of ordinary skill in the art will be aware of suitable methods for determining such characteristics, for example, commercially-available UV detectors, fluorescent detectors, thermocouples, or the like.

In any of the embodiments described herein, a droplet may be directed to a particular region of a device, e.g., for sorting or screening purposes, based at least in part upon the determining of the droplet, as discussed above. For example, a characteristic of a fluid droplet may be determined in some fashion, for example, as described above (e.g., fluorescence), and, in response, an electric field may be imposed or removed to direct the droplet to a particular region (e.g. an exit channel) within a device of the invention, for instance, to chambers 132 or 134 as illustrated in FIG. 1. The electric field may cause the droplet to move to a particular channel or region due to electric field attraction, electric field repulsion, dielectrophoresis, etc. As another example, the interaction of the agent and the cell and/or other species within the droplet may result in the placement of an electric charge on the droplet which can be subsequently directed using an electric field. Systems and methods for screening and/or sorting droplets are disclosed in, for example, U.S. patent application Ser. No. 11/360,845, filed Feb. 23, 2006, entitled “Electronic Control of Fluidic Species,” published as U.S. Patent Application Publication No. 2007/000342 on Jan. 4, 2007, incorporated herein by reference.

As a particular example, one or more cells may be exposed to a marker or other agent, such as a fluorescent composition, that binds or otherwise associates with the cell if a certain condition is present. For example, the agent may bind or otherwise associate if a species (e.g., a sugar) is metabolized to or beyond a certain extent, a protein is expressed, or the marker may bind to a first cell type but not a second cell type. For example, a marker may bind to first cell type 116, but not second cell type 117 in FIG. 1. In some cases, the agent may be indicative of the viability of the cell (e.g., whether the cell is alive or dead), the state of development or differentiation of the cell, etc. The cells may be directed based on the presence and/or magnitude of the agent. For instance, detection of a fluorescent signal may cause the cells of a first type to be directed to one region of the device (e.g., optional storage chamber 132 in FIG. 1), while the absence of the fluorescent signal may cause the cells of a second type to be directed to another region of the device (e.g., optional waste chamber 134 in FIG. 1). Thus, in this example, a population of cells may be screened and/or sorted on the basis of one or more detectable or targetable characteristics of the cells, for example, to select live cells, cells exhibiting certain growth rates, cells expressing a certain protein, a certain cell type, etc. In some embodiments, the DNA of one or more of the cell types may be sequenced, as discussed in U.S. Pat. Apl. Ser. No. 61/008,862, filed Dec. 21, 2007, incorporated herein by reference.

After sorting, the cells may then be collected, e.g., to produce an enriched population of cells. For instance, FIG. 1 shows an optional storage chamber 132. The storage chamber may comprise any compartment capable of holding droplets. In some embodiments, the storage chamber may be integral to the device comprising microfluidic channel 110, while in other embodiments, the storage chamber may be removable (e.g., a removable syringe, an external container, etc.). As used herein, “integral” means that portions of the components integral to each other are joined in such a way that the components cannot be manually separated from each other without the use of tools, or without cutting or breaking at least one of the components.

While FIG. 1 illustrates an embodiment including two cell types in chamber 130, it should be understood that three, four, or more cell types may be contained in chamber 130 in some cases, and in some instances, two or more chambers may be present. In some embodiments, it is not known a priori how many cell types and/or the number of cells of each type are present in the population of droplets in chamber 130. In addition, in other embodiments, other species may be used besides cells, as previously noted.

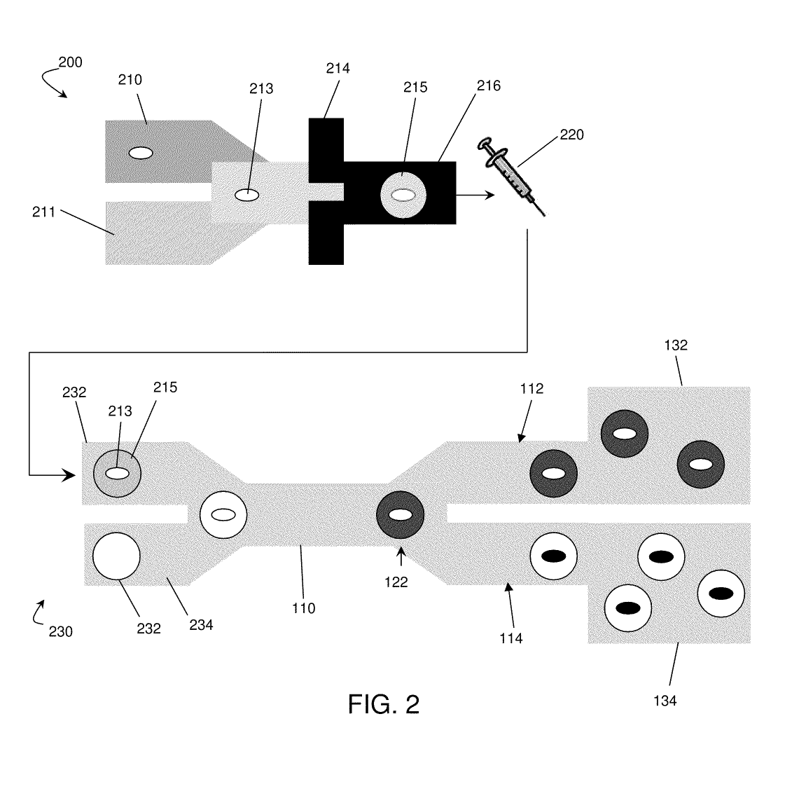

Thus, the present invention is not limited to the device as is shown in FIG. 1, but includes other systems and methods as well. For instance, FIG. 2 includes a schematic diagram outlining a method of culturing and/or sorting cells or other species, as another example. In this set of embodiments, a first device 200 comprises a cell channel 210 and a culture media channel 211 which converge to form a single channel. Upon convergence, the cell carrier phase from the cell channel and the culture media mix to form a combined continuous phase surrounding cells 213. In some embodiments, the culture media and/or the cell carrier phase comprises one or more agents capable of reacting with the cells. For example, in some embodiments, the one or more agents may comprise a sugar (e.g., xylose, deoxyribose, sucrose, fructose, etc.) or other carbohydrate. The combined continuous phase can be passed through secondary carrier channel 214. The secondary carrier channel is used to inject a secondary carrier phase that is immiscible with the combined continuous phase surrounding the cells. As the combined carrier phase is injected through carrier channel 214, droplets 215 are formed.

Systems and methods for forming droplets suitable for use in systems such as those described above are described in various documents, including U.S. patent application Ser. No. 11/360,845, filed Feb. 23, 2006, entitled “Electronic Control of Fluidic Species,” published as U.S. Patent Application Publication No. 2007/000342 on Jan. 4, 2007, International Patent Application No. PCT/US2006/007772, filed Mar. 3, 2006, entitled “Method and Apparatus for Forming Multiple Emulsions,” published as WO 2006/096571 on Sep. 14, 2006, each incorporated herein by reference. In some instances, these droplets may contain at least one cell (or other species), while in other instances, the droplets may not contain any cells or species.

In the example of FIG. 2, the droplets are collected from exit channel 216, and accumulated in storage container 220. The storage container comprises a syringe in this example. In other embodiments, the storage container may be any other container capable of holding the droplets. In some cases, the storage container may be integral to device 200 (e.g., a well within the device), while in other cases, the storage container may be separated (e.g., a bag, a vial, a microwell plate, a bottle, a tank, etc. that is physically separated from the device). Once droplets have been accumulated, the droplets may be collected and cultured (if the droplets contain cells) in the storage container (e.g., by transfer to an incubator). For example, in cases where a syringe is used, the syringe may be, for instance, capped and moved to an incubator. The droplets may be cultured for any suitable period of time including, for example, at least 1 hour, at least about 2 hours, at least about 3 hours, at least 8 hours, at least 1 day, at least 2 days, at least 3 days, at least 7 days, at least 1 week, at least 1 month, etc.

Referring again to the example shown in FIG. 2, the cells in the storage container may be injected into device 230 via droplet inlet channel 232. In some embodiments, device 230 is integral to device 200 on a single substrate. In other embodiments, device 230 and device 200 are physically separated. Optionally, agent inlet channel 234 may be used to inject agent droplet 232, which may contain one or more additional agents that may interact with cell 213. In cases where a second agent is employed, agent droplet 232 may coalesce with droplet 215. In some embodiments, coalescence may occur upon the application of an electromagnetic field. In other cases, coalescence may occur without an outside stimulus.

Systems and methods for use in the coalescence of droplets are described in U.S. patent application Ser. No. 11/360,845, filed Feb. 23, 2006, entitled “Electronic Control of Fluidic Species,” published as U.S. Patent Application Publication No. 2007/000342 on Jan. 4, 2007, or in U.S. patent application Ser. No. 11/698,298, filed Jan. 24, 2007, entitled “Fluidic Droplet Coalescence,” each incorporated herein by reference. In some instances, instead of or in addition to introducing a second agent via agent droplet 232, second agent may be introduced in the continuous phase of channel 110. In some cases, at least some of the droplets are exposed to a second agent for a time at least sufficient to allow the second agent to enter at least some of the droplets.

The extent of reaction between an agent and the cell and/or other species within the droplet may also be determined, for example, at some point within a channel. In some cases, the channel may be made fairly long (e.g., serpentine), for instance, to increase the amount of time a reaction can occur before a determination is made. As a specific example in the set of embodiments illustrated in FIG. 2, the determination is made at point 122. Examples of characteristics determinable within the droplet and usable in the invention may be identified by those of ordinary skill in the art, and include but are not limited to, for example, fluorescence, spectroscopy (e.g., optical, infrared, ultraviolet, etc.), radioactivity, mass, volume, density, temperature, viscosity, pH, concentration of a substance, such as a biological substance (e.g., a protein, a nucleic acid, etc.), viability of one or more cells within the droplet, or the like. The set of embodiments illustrated in FIG. 2 may be used to produce an enriched population of cells.

The droplets can be formed in one step in some cases, often with precise repeatability, and can be tailored to include one, two, three or more cells within a single droplet. The term “droplet,” as used herein, refers to an isolated portion of a first fluid that is surrounded by a second fluid, where the first and second fluids are immiscible on the time scale of use of the device of the invention (e.g., the time it takes a fluid droplet to flow through a particular system or device). As used herein, the term “fluid” generally refers to a substance that tends to flow and to conform to the outline of its container. Typically, fluids are materials that are unable to withstand a static shear stress, and when a shear stress is applied, the fluid experiences a continuing and permanent distortion. The fluid may have any suitable viscosity that permits at least some flow of the fluid. Non-limiting examples of fluids include liquids and gases, but may also include free-flowing solid particles (e.g., cells, vesicles, etc.), viscoelastic fluids, and the like. Making and using such droplets, including use in a variety of chemical, biological or biochemical settings, and techniques for encapsulating cells within such droplets, are described in various documents, including International Patent Application No. PCT/US2006/007772, filed Mar. 3, 2006, entitled “Method and Apparatus for Forming Multiple Emulsions,” published as WO 2006/096571 on Sep. 14, 2006, or in International Patent Application No. PCT/US2004/010903, filed Apr. 9, 2004, entitled “Formation and Control of Fluidic Species,” published as WO 2004/091763 on Oct. 28, 2004, each incorporated herein by reference.

In certain instances, the droplets may be contained within a carrying fluid, e.g., within a fluidic stream. The fluidic stream, in one set of embodiments, is created using a to microfluidic system, discussed in detail below. In some cases, the droplets will have a homogenous distribution of diameters, i.e., the droplets may have a distribution of diameters such that no more than about 10%, about 5%, about 3%, about 1%, about 0.03%, or about 0.01% of the droplets have an average diameter greater than about 10%, about 5%, about 3%, about 1%, about 0.03%, or about 0.01% of the average diameter of the droplets. Techniques for producing such a homogenous distribution of diameters are also disclosed in International Patent Application No. PCT/US2004/010903, filed Apr. 9, 2004, entitled “Formation and Control of Fluidic Species,” by Link, et al., published as WO 2004/091763 on Oct. 28, 2004, incorporated herein by reference, and in other references as described below.

The devices and methods described herein may be used for high-throughput screening and/or sorting of cells and/or droplets. In some cases, at least about 10 droplets per second may be determined and/or sorted in such a fashion, and in other cases, at least about 20 droplets per second, at least about 30 droplets per second, at least about 100 droplets per second, at least about 200 droplets per second, at least about 300 droplets per second, at least about 500 droplets per second, at least about 750 droplets per second, at least about 1000 droplets per second, at least about 1500 droplets per second, at least about 2000 droplets per second, at least about 3000 droplets per second, at least about 5000 droplets per second, at least about 7500 droplets per second, at least about 10,000 droplets per second, at least about 15,000 droplets per second, at least about 20,000 droplets per second, at least about 30,000 droplets per second, at least about 50,000 droplets per second, at least about 75,000 droplets per second, at least about 100,000 droplets per second, at least about 150,000 droplets per second, at least about 200,000 droplets per second, at least about 300,000 droplets per second, at least about 500,000 droplets per second, at least about 750,000 droplets per second, at least about 1,000,000 droplets per second, at least about 1,500,000 droplets per second, at least about 2,000,000 or more droplets per second, or at least about 3,000,000 or more droplets per second may be determined and/or sorted in such a fashion.

In some embodiments, as previously described, the fluid droplets may be screened or sorted within a device of the invention without substantially altering the flow characteristics of the liquid containing the droplets, e.g., without the use of any mechanical flow-controlling devices such as valves, pumps, pistons, etc. For example, a liquid may flow on a substantially steady-state basis (i.e., substantially unchanging with respect to time) or other predetermined basis through a device, and fluid droplets within the liquid may be directed to various locations within the device using an electrical field as previously described, without substantially altering the flow of the liquid.

In one set of embodiments, in a plurality of droplets of fluid, some of which droplets contain a species of interest and some of which droplets do not contain the species of interest, the droplets of fluid may be screened or sorted for those droplets of fluid containing the species, and in some cases, the droplets may be screened or sorted for those droplets of fluid containing a particular number or range of entities of the species of interest. Systems and methods for screening and/or sorting droplets are disclosed in, for example, U.S. patent application Ser. No. 11/360,845, filed Feb. 23, 2006, entitled “Electronic Control of Fluidic Species,” published as U.S. Patent Application Publication No. 2007/000342 on Jan. 4, 2007, incorporated herein by reference.

Thus, in some cases, a plurality or series of fluidic droplets, some of which droplets contain the species and some of which droplets do not, may be enriched (or depleted) in the ratio of droplets that do contain the species, for example, by a factor of at least about 2, at least about 3, at least about 5, at least about 10, at least about 15, at least about 20, at least about 50, at least about 100, at least about 125, at least about 150, at least about 200, at least about 250, at least about 500, at least about 750, at least about 1000, at least about 2000, or at least about 5000 or more in some cases. In other cases, the enrichment (or depletion) may be in a ratio of at least about 10⁴, at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, at least about 10¹⁵, or more. For example, a fluidic droplet containing a particular species may be selected from a library of fluidic droplets containing various species, where the library may have about 10⁵, about 10⁶, about 10⁷, about 10⁸, about 10⁹, about 10¹⁰, about 10¹¹, about 10¹², about 10¹³, about 10¹⁴, about 10¹⁵, or more items, for example, a DNA library, an RNA library, a protein library, a combinatorial chemistry library, etc. In certain embodiments, the droplets carrying the species may then be fused, reacted, or otherwise used or processed, etc., as further described herein, for example, to initiate or determine a reaction.

In some, but not all embodiments, all components of the systems and methods described herein are microfluidic. “Microfluidic,” as used herein, refers to a device, apparatus or system including at least one fluid channel having a cross-sectional dimension of less than 1 mm, and a ratio of length to largest cross-sectional dimension of at least 3:1. A “microfluidic channel,” as used herein, is a channel meeting these criteria.

The “cross-sectional dimension” of the channel is measured perpendicular to the direction of fluid flow. Most fluid channels in components of the invention have maximum cross-sectional dimensions less than 2 mm, and in some cases, less than 1 mm. In one set of embodiments, all fluid channels containing embodiments of the invention are microfluidic or have a largest cross sectional dimension of no more than 2 mm or 1 mm. In another embodiment, the fluid channels may be formed in part by a single component (e.g. an etched substrate or molded unit). Of course, larger channels, tubes, chambers, reservoirs, etc. can be used to store fluids in bulk and to deliver fluids to components of the invention. In one set of embodiments, the maximum cross-sectional dimension of the channel(s) containing embodiments of the invention are less than 500 microns, less than 200 microns, less than 100 microns, less than 50 microns, or less than 25 microns.

A “channel,” as used herein, means a feature on or in an article (substrate) that at least partially directs the flow of a fluid. The channel can have any cross-sectional shape (circular, oval, triangular, irregular, square or rectangular, or the like) and can be covered or uncovered. In embodiments where it is completely covered, at least one portion of the channel can have a cross-section that is completely enclosed, or the entire channel may be completely enclosed along its entire length with the exception of its inlet(s) and outlet(s). A channel may also have an aspect ratio (length to average cross sectional dimension) of at least 2:1, more typically at least 3:1, 5:1, or 10:1 or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to fluid flow of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases the dimensions of the channel may be chosen such that fluid is able to freely flow through the article or substrate. The dimensions of the channel may also be chosen, for example, to allow a certain volumetric or linear flowrate of fluid in the channel. Of course, the number of channels and the shape of the channels can be varied by any method known to those of ordinary skill in the art.

As used herein, a first entity is “surrounded” by a second entity if a closed loop can be drawn around the first entity through only the second entity. A first entity is “completely surrounded” if closed loops going through only the second entity can be drawn around the first entity regardless of direction. In one aspect, the first entity may be a cell, for example, a cell suspended in media is surrounded by the media. In another aspect, the first entity is a particle. In yet another aspect of the invention, the entities can both be fluids. For example, a hydrophilic liquid may be suspended in a hydrophobic liquid, a hydrophobic liquid may be suspended in a hydrophilic liquid, a gas bubble may be suspended in a liquid, etc. Typically, a hydrophobic liquid and a hydrophilic liquid are substantially immiscible with respect to each other, where the hydrophilic liquid has a greater affinity to water than does the hydrophobic liquid. Examples of hydrophilic liquids include, but are not limited to, water and other aqueous solutions comprising water, such as cell or biological media, ethanol, salt solutions, etc. Examples of hydrophobic liquids include, but are not limited to, oils such as hydrocarbons, silicon oils, fluorocarbon oils, organic solvents etc.

The term “determining,” as used herein, generally refers to the analysis or measurement of a species, for example, quantitatively or qualitatively, or the detection of the presence or absence of the species. “Determining” may also refer to the analysis or measurement of an interaction between two or more species, for example, quantitatively or qualitatively, or by detecting the presence or absence of the interaction. Example to techniques include, but are not limited to, spectroscopy such as infrared, absorption, fluorescence, UV/visible, FTIR (“Fourier Transform Infrared Spectroscopy”), or Raman; gravimetric techniques; ellipsometry; piezoelectric measurements; immunoassays; electrochemical measurements; optical measurements such as optical density measurements; circular dichroism; light scattering measurements such as quasielectric light scattering; polarimetry; refractometry; or turbidity measurements.

In cases where multiple droplets are present, the droplets may each be substantially the same shape and/or size (“monodisperse”). The shape and/or size of the droplets can be determined, for example, by measuring the average diameter or other characteristic dimension of the droplets. The average diameter of a droplet (and/or of a plurality or series of droplets) may be, for example, less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers in some cases. The average diameter may also be at least about 1 micrometer, at least about 2 micrometers, at least about 3 micrometers, at least about 5 micrometers, at least about 10 micrometers, at least about 15 micrometers, at least about 20 micrometers, or at least about 100 micrometers in certain cases.

The “average diameter” of a population of droplets is the arithmetic average of the diameters of the droplets. Those of ordinary skill in the art will be able to determine the average diameter of a population of droplets, for example, using laser light scattering or other known techniques. The diameter of a droplet, in a non-spherical droplet, is the mathematically-defined average diameter of the droplet, integrated across the entire surface. As non-limiting examples, the average diameter of a droplet may be less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers. The average diameter of the droplet may also be at least about 1 micrometer, at least about 2 micrometers, at least about 3 micrometers, at least about 5 micrometers, at least about 10 micrometers, at least about 15 micrometers, or at least about 20 micrometers in certain cases.

A variety of materials and methods can be used to form components of the system, in certain aspects of the invention. In some cases various materials selected lend themselves to various methods. For example, components of the invention can be formed from solid materials, in which the channels can be formed via micromachining, film deposition processes such as spin coating and chemical vapor deposition, laser fabrication, photolithographic techniques, etching methods including wet chemical or plasma processes, and the like. See, for example, Angell, et al., Scientific American 248:44-55 (1983). In one embodiment, at least a portion of the system is formed of silicon by etching features in a silicon chip. Technology for precise and efficient fabrication of devices of the invention from silicon is known. In another embodiment that section (or other sections) can be formed of a polymer, and can be an elastomeric polymer, or polytetrafluoroethylene (PTFE, Teflon®), or the like.

Different components can be fabricated of different materials. For example, a base portion including a bottom wall and side walls can be fabricated from an opaque material such as silicon, and a top portion can be fabricated from a transparent material such as glass or a transparent polymer, for observation and control of the fluidic process. Components can be coated so as to expose a desired chemical functionality to fluids that contact interior channel walls, where base supporting material does not have the precise, desired functionality. For example, components can be fabricated as illustrated, with interior channel walls coated with another material.

Material used to fabricate devices of the invention, or material used to coat interior walls of fluid channels, may desirably be selected from among those materials that will not adversely affect or be affected by fluid flowing through the device, e.g., material(s) that is chemically inert in the presence of fluids to be used within the device. Non-limiting examples of such coatings are disclosed in U.S. Pat. Apl. Ser. No. 61/040,442, filed Mar. 28, 2008, entitled “Surfaces, Including Microfluidic Channels, With Controlled Wetting Properties,” incorporated herein by reference.

In one embodiment, components of the invention are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid art that can be induced to solidify, or that spontaneously solidifies, into a solid capable of containing and transporting fluids contemplated for use in and with the network to structure. In one embodiment, the hardenable fluid comprises a polymeric liquid or a liquid polymeric precursor (i.e. a “prepolymer”). Suitable polymeric liquids can include, for example, thermoplastic polymers, thermoset polymers, or mixture of such polymers heated above their melting point; or a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Examples of silicone elastomers suitable for use according to the invention include those formed from precursors including the chlorosilanes such as methylchlorosilanes, ethylchlorosilanes, and phenylchlorosilanes, and the like.

Silicone polymers are preferred in one set of embodiments, for example, the silicone elastomer poly(dimethylsiloxane) (PDMS). Exemplary polydimethylsiloxane polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of the microfluidic structures of the invention. For instance, such materials are inexpensive, readily available, and can be solidified from a prepolymeric liquid via curing with heat. For example, PDMSs are typically curable by exposure of the prepolymeric liquid to temperatures of about, for example, 65° C. to about 75° C. for exposure times of about, for example, 1 hour. Also, silicone polymers, such as PDMS, can be elastomeric and thus may be useful for forming very small features with relatively high aspect ratios, necessary in certain embodiments of the invention. Flexible (e.g. elastomeric) molds or masters can be advantageous in this regard.

One advantage of forming structures such as microfluidic structures of the invention from silicone polymers, such as PDMS, is the ability of such polymers to be oxidized, for example by exposure to an oxygen-containing plasma such as an air plasma, so that the oxidized structures contain at their surface chemical groups capable of cross-linking to other oxidized silicone polymer surfaces or to the oxidized surfaces of a variety of other polymeric and non-polymeric materials. Thus, components can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in Duffy et al., Rapid Prototyping of Microfluidic Systems and Polydimethylsiloxane, Analytical Chemistry, Vol. 70, pages 474-480, 1998, incorporated herein by reference.

Another advantage to forming microfluidic structures of the invention (or interior, fluid-contacting surfaces) from oxidized silicone polymers is that these surfaces can be much more hydrophilic than the surfaces of typical elastomeric polymers (where a hydrophilic interior surface is desired). Such hydrophilic channel surfaces can thus be more easily filled and wetted with aqueous solutions than can structures comprised of typical, unoxidized elastomeric polymers or other hydrophobic materials.

In one embodiment, a bottom wall is formed of a material different from one or more side walls or a top wall, or other components. For example, the interior surface of a bottom wall can comprise the surface of a silicon wafer or microchip, or other substrate. Other components can, as described above, be sealed to such alternative substrates. Where it is desired to seal a component comprising a silicone polymer (e.g. to PDMS) to a substrate (bottom wall) of different material, it is preferred that the substrate be selected from the group of materials to which oxidized silicone polymer is able to irreversibly seal (e.g., glass, silicon, silicon oxide, quartz, silicon nitride, polyethylene, polystyrene, epoxy polymers, and glassy carbon surfaces which have been oxidized). Alternatively, other sealing techniques can be used, as would be apparent to those of ordinary skill in the art, including, but not limited to, the use of separate adhesives, thermal bonding, solvent bonding, ultrasonic welding, etc.

Certain aspects of the invention are directed to kits including one or more of the devices discussed above. A “kit,” as used herein, typically defines a package or an assembly including one or more of the apparatuses of the invention, and/or other apparatuses associated with the invention, for example, as previously described. A kit of the invention may, in some cases, include instructions in any form that are provided in connection with the apparatuses of the invention in such a manner that one of ordinary skill in the art would recognize that the instructions are to be associated with the apparatuses of the invention. For instance, the instructions may include instructions for the use, modification, assembly, storage, packaging, and/or preparation of the apparatuses and/or other apparatuses associated with the kit. In some cases, the instructions may also include instructions, for example, for a particular use, e.g., to a sample. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

One aspect of the invention provides for methods of promoting one or more of the embodiments disclosed herein. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, apparatuses, methods, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

The following applications are each incorporated herein by reference: U.S. Provisional Patent Application Ser. No. 61/076,473, filed Jun. 27, 2008, entitled “Microfluidic Droplets for Metabolic Engineering and Other Applications,” by Wang, et al.; U.S. patent application Ser. No. 08/131,841, filed Oct. 4, 1993, entitled “Formation of Microstamped Patterns on Surfaces and Derivative Articles,” by Kumar, et al., now U.S. Pat. No. 5,512,131, issued Apr. 30, 1996; U.S. patent application Ser. No. 09/004,583, filed Jan. 8, 1998, entitled “Method of Forming Articles including Waveguides via Capillary Micromolding and Microtransfer Molding,” by Kim, et al., now U.S. Pat. No. 6,355,198, issued Mar. 12, 2002; International Patent Application No. PCT/US96/03073, filed Mar. 1, 1996, entitled “Microcontact Printing on Surfaces and Derivative Articles,” by Whitesides, et al., published as WO 96/29629 on Jun. 26, 1996; International Patent Application No.: PCT/US01/16973, filed May 25, 2001, entitled “Microfluidic Systems including Three-Dimensionally Arrayed Channel Networks,” by Anderson, et al., published as WO 01/89787 on Nov. 29, 2001; U.S. patent application Ser. No. 11/246,911, filed Oct. 7, 2005, entitled “Formation and Control of Fluidic Species,” by Link, et al., published as U.S. Patent Application Publication No. 2006/0163385 on Jul. 27, 2006; U.S. patent application Ser. No. 11/024,228, filed Dec. 28, 2004, entitled “Method and Apparatus for Fluid Dispersion,” by Stone, et al., published as U.S. Patent Application Publication No. 2005/0172476 on Aug. 11, 2005; International Patent Application No. PCT/US2006/007772, filed Mar. 3, 2006, entitled “Method and Apparatus for Forming Multiple Emulsions,” by Weitz, et al., published as WO 2006/096571 on Sep. 14, 2006; U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007; and U.S. Patent application Ser. No. 11/368,263, filed Mar. 3, 2006, entitled “Systems and Methods of Forming Particles,” by Garstecki, et al.

The following examples are intended to illustrate certain embodiments of the present invention, but do not exemplify the full scope of the invention.

### Example 1

This example describes a high-throughput screening platform which utilizes microfluidics to encapsulate yeast cells in nanoliter aqueous droplets surrounded by an immiscible fluorinated oil phase. The system described in this example contained modules for cell culturing, measurement of the metabolite of interest with a fluorescent enzymatic assay, and sorting. In this example, a population of high xylose-consuming cells was enriched by over 21 times from a mixture of two Saccharomyces cerevisiae (yeast) strains. This systems and methods described in this example can be expanded to apply more generally to select for strains from libraries for metabolic engineering applications. Any fluorescent assay system can be used in this example. Furthermore, the enzymes described here (e.g., horseradish peroxidase/Amplex UltraRed) are by way of example only and other enzymes, such as other oxidase enzymes which exist in nature, can be used as well.

This example involves the consumption of xylose by S. cerevisiae, which has received attention, for instance, in the field of biofuels. Lignocellulosic feedstocks, such as corn stover, contains a significant amount of xylose. However, S. cerevisiae, which readily converts glucose to ethanol, cannot naturally ferment xylose. As a result, engineering a S. cerevisiae strain which can readily utilize xylose is an important step in developing various lignocellulosic ethanol process.

Two S. cerevisiae strains were used in this example, which will be referred to here as H131 and TAL1. H131 was derived from F1702 (a BF-264-15Daub derivative). F1702 is H131 is MAT_a, leu2, ura3, arg4, ade1, trp1, his2. The genotype of H131 is to MAT_a, leu2, ura3, arg4, ade1::ADE1-GPD-PsTAL1, trp1::TRP1-GPDp-ScRKI1-ScRPE1, his2::HIS2-GPD_{P}-ScTKL1 with a the plasmid PRS426-GPD_{P}-XYL1-XYL2-XYL3-CYC_{T}. TAL1 was created from YSX3 which had a genotype of is MAT_alpha, trp1, leu2::LEU2-GAPDHP-XYL1 ura3::URA3-GAPDHP-XYL2 Ty3::NEO-XYL3. TAL1 is YSX3 with the pRS424TEF-PsTAL1 plasmid.

For the quantification of xylose concentrations, cell-free culture supernatants were filtered through 0.2 micrometer pore-size polytetrafluoroethylene membrane syringe filters (VWR International) and used for high-performance liquid chromatography (HPLC) analysis with a Waters 2690 Separations module connected with a Waters 410 refractive index detector (Waters). The samples were separated on a BioRad Aminex HPX-87H ion exclusion column for organic acid analysis with 14 mM sulfuric acid as the mobile phase at a flow rate of 0.7 mL/min. For cell density determinations, the optical densities of cultures and cell-free culture supernatants were measured at 600 nm using an Ultrospec 2100 pro UV/visible spectrophotometer (Amersham Biosciences). The assay mixture contained phosphate buffered saline (PBS), Amplex UltraRed (Invitrogen), pyranose oxidase (Sigma), and horseradish peroxidase (Sigma). The concentration of the components in the assay droplets were 4 U/mL pyranose oxidase, 0.4 U/mL horseradish peroxidase, and 0.2 mM Amplex UltraRed. The tubing used to supply the assay reagents to the device was pretreated with 1% bovine serum albumin for 5 minutes so that components in the assay mixture did not adhere to the tubing.

Microfluidic devices were fabricated by using standard, well-known soft lithography techniques, for example, as discussed in Int. Pat. Apl. Pub. No. WO 97/33737, published on Sep. 18, 1997, incorporated herein by reference. SU-8 2025 and 2050 photoresists (MicroChem) were spin coated at either a 25 or 75 micrometer thickness on 3 inch test grade silicon wafers. The channel pattern was photolithographically defined by a film mask printed at 20,000 dpi (dots per inch). After developing the photoresist, poly(dimethylsiloxane) (PDMS) (Sylgard 184 Silicone Elastomer Kit from Dow Chemical) in a 10:1 silicone to crosslinker ratio was poured onto the wafer. After degassing for 10 minutes and curing overnight at 65° C., the devices were cut from the mold, the inlets and outlets were formed with a biopsy punch, and bonded to glass slides with an oxygen plasma. The 2 inch×3 inch glass slides for to devices with electrodes contained an indium tin oxide coating (Delta Technologies) on the surface opposite the device. Electrodes were also fabricated in the device. The electrode channels were first coated with 0.1 M (3-mercaptopropyl)trimethoxysilane (Sigma) dissolved in acetonitrile (Sigma) and air was then blown to remove the solution. The device was then baked at 65° C. to remove any remaining solution. When the device was placed on a hot plate at 80° C., Indalloy 19 solder (52% In, 32.5% Bi, 16.5% Sn-0.020 inch diameter wire from Indium Corporation) was placed in the electrode inlets and allowed to melt. Once the solder reached the outlets, 22 gauge wire was placed in the outlets. All other devices used plain 2 inch×3 inch Swiss Glass slides. Before using the devices, the PDMS channel surface was made hydrophobic by injecting Aquapel (PPG) into the channels and then blowing air to remove the Aquapel.

Liquids were supplied to microfluidic device by syringes connected to NE-500 syringe pumps (New Era) with PE-20 (Intramedic) or PEEK (VICI Valco) tubing. Proprietary fluorinated oil and surfactants were supplied by Raindance Technologies, although any suitable fluorinated oil and/or surfactant could also be used, such as those disclosed in Int. Pat. Apl. Pub. No. WO 2008/021123, published on Feb. 21, 2008, incorporated herein by reference. To monitor the functionality of microfluidic device, an Ultima 512 (Photron) was connected to a Motic AE30 inverted microscope. The coalescence electrodes were connected to an inverter and DC power supply while the sorting electrodes were connected to a high voltage amplifier (TREK). Fluorescence detection was performed using a 50 mW 488 nm laser (Picarro) for excitation and a photomultiplier tube (Hamamatsu) with a 593 nm filter (Semrock). Syringes were incubated in a Lab-Line compact incubator.

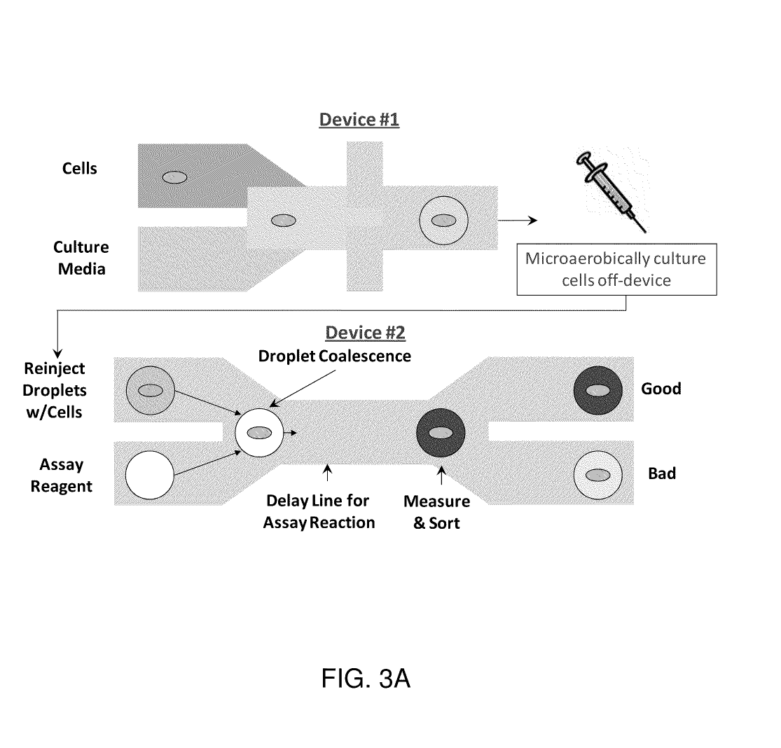

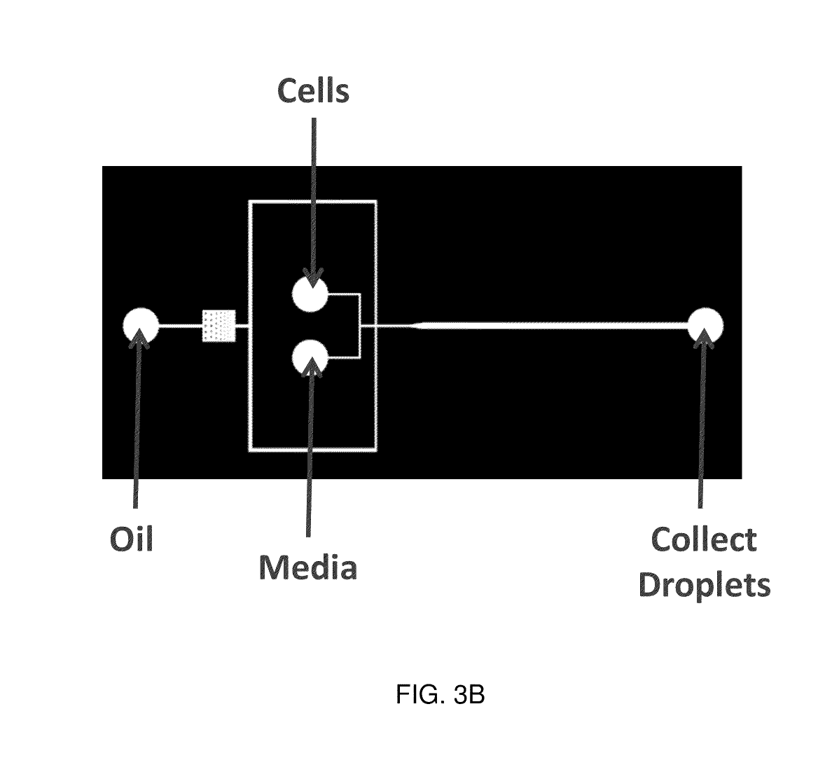

The high-throughput screening platform in this example has the capability to compartmentalize droplets containing yeast cells, culture the cells, mix the contents of the droplets containing cells with a fluorescent enzymatic assay, measure the resulting fluorescence, and sort droplets based on that measurement. In order to perform all of these functions, two microfluidic devices and a syringe were utilized as shown in FIG. 3A (in other embodiments, however, the microfluidic devices may be combined together). The first device was used to mix yeast cells in PBS with the cell culture media and form droplets by combining that aqueous stream with two streams containing a fluorinated oil and surfactant mixture. The droplets formed in this device were collected in a syringe. Once the syringe was full, it was capped to prevent air from coming into contact with the droplets to allow for microaerobic culturing. The syringe was then placed in an incubator at 30° C. to culture the cells. After a predetermined amount of time, the syringes were used to reinject the droplets into a second device in which they were combined with another set of droplets containing fluorescent enzymatic assay reagents. After droplet coalescence, the resulting droplets were flowed through long channels for 30 seconds to allow the assay reaction to proceed, after which a laser and photomultiplier tube system was used for fluorescent excitation and emission detection measurement. Based on this measurement, the droplets were sorted into one of two “bins” (although more than two bins may be used in other cases).

In order to demonstrate the functionality of this platform, the consumption of xylose by Saccharomyces cerevisiae was chosen to be the screening metric. Portions of the devices were initially tested individually to ensure that they worked before combining them together into a complete device.

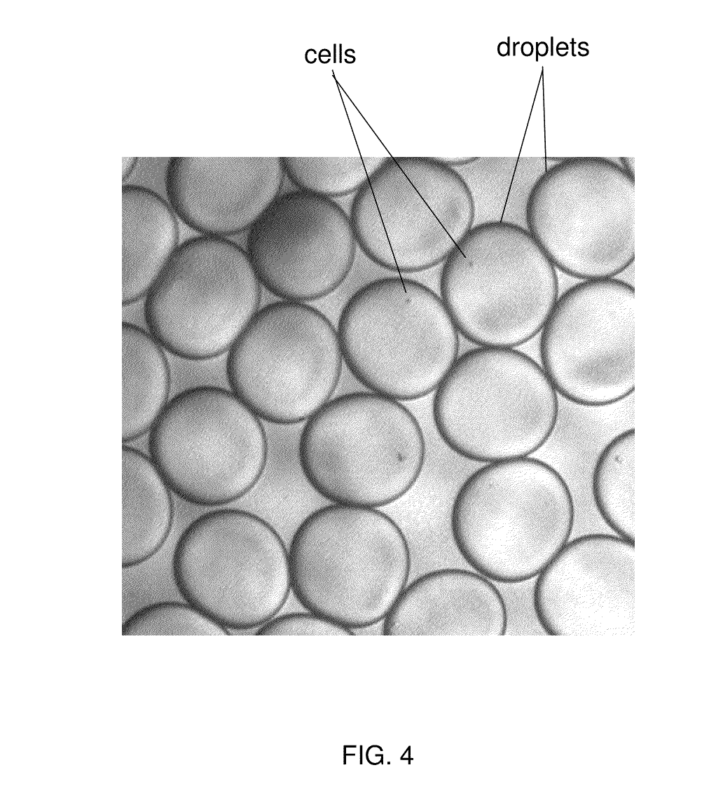

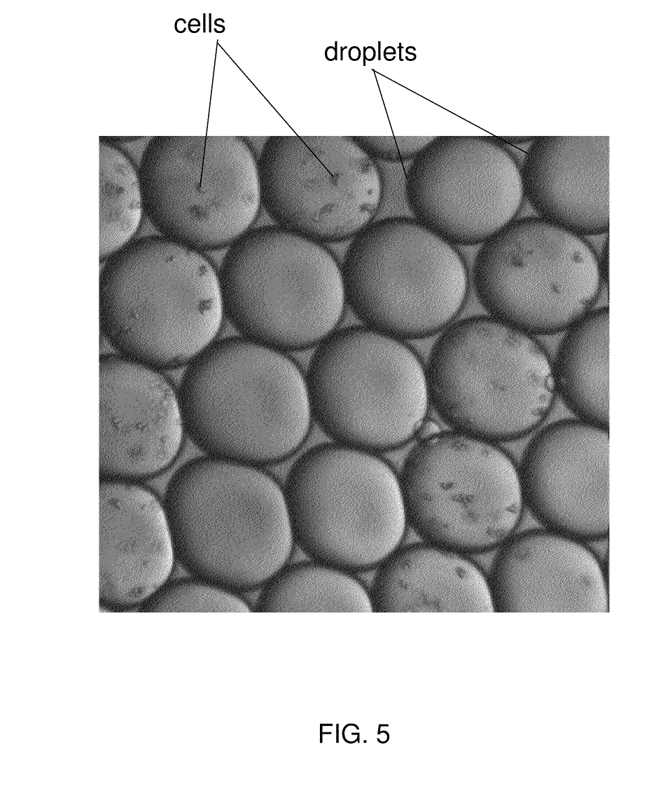

A simple coflow droplet maker was used to create droplets containing yeast cells as shown in FIG. 3B. The channels in this device were 25 micrometers tall. The droplets formed by this device were approximately 90 micrometers in diameter, resulting in a volume of less than 1 nL. In order to encapsulate one cell in approximately every three droplets, the OD600 cell density of the incoming cells was 0.075. FIG. 4 shows individual cells in droplets. The droplets formed in the droplet maker were collected in a syringe and cultured microaerobically by capping the syringe. FIG. 5 shows droplets containing TAL1 cells cultured after 3.5 days.

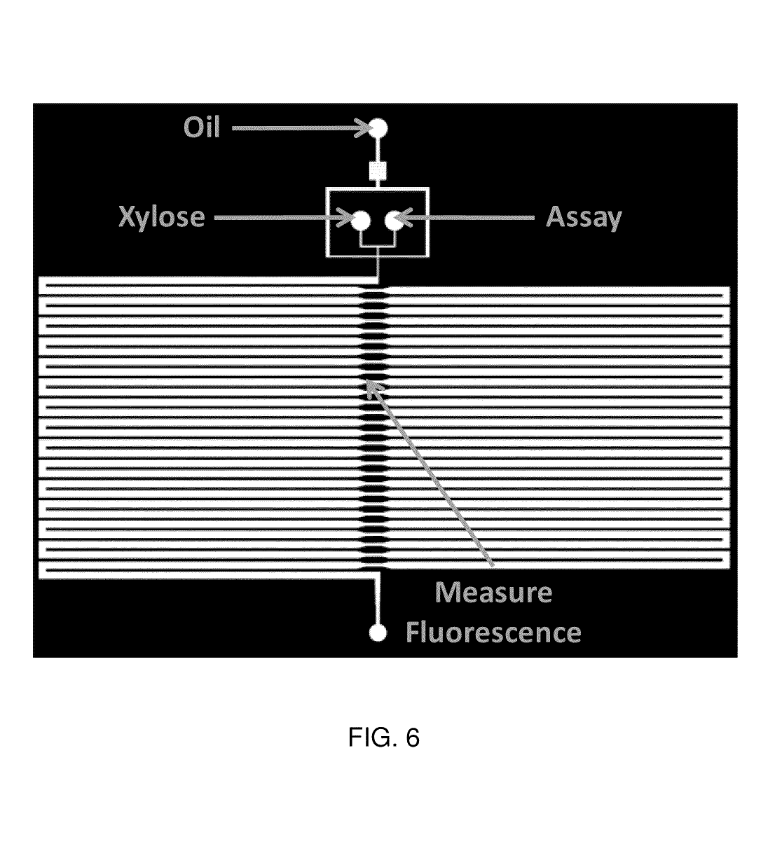

The first portion of the second device that was tested was the assay reaction in droplets through the delay lines. A schematic of the microfluidic device used for testing is shown in FIG. 6. The channels in this device were 75 micrometers tall. This device mixed two aqueous inputs, one containing xylose and another containing the assay mixture. Generally, the assay reaction may be represented as:

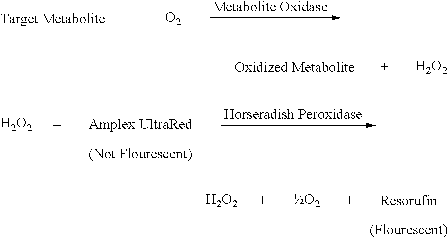

The assay used to detect xylose contained 2 U/mL pyranose oxidase, 0.4 U/mL horseradish peroxidase, and 0.2 mM Amplex UltraRed (Molecular Probes). The assay reaction is shown below:

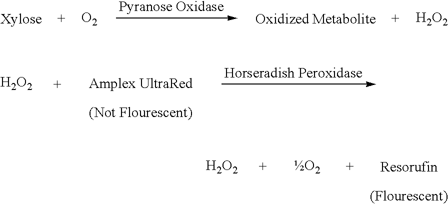

The amount of fluorescent resorufin produced was proportional to the concentration of xylose in solution.

The tubing used to supply the assay reagents to the device was pretreated with 1% bovine serum albumin for 5 minutes so that components in the assay mixture did not adhere to the tubing, which would decrease the actual concentration supplied to the device. Droplets were formed when the aqueous stream (a mixture of xylose and the assay reagents discussed above) came into contact with the oil stream. Then, the droplets flowed through long microfluidic channel delay lines to allow the assay to proceed. Microfluidic delay lines were chosen instead of tubing because it was observed that the fluorescence distribution was not as tight when tubing was used. This device was designed so that fluorescence could be measured at different locations along the delay line to determine the optimal measurement point.

Several experiments were run using this device in which the xylose concentration was varied. The fluorescence increased with increasing xylose concentration, and the resulting fluorescence distributions were relatively narrow. The correlation between fluorescence and xylose concentration was also relatively linear, and could be described by the following equation:

Fluorescence=(0.1879*Xylose Concentration)+0.1211 [1]

The correlation in Equation 1 resulted in an R² value of 0.9455.

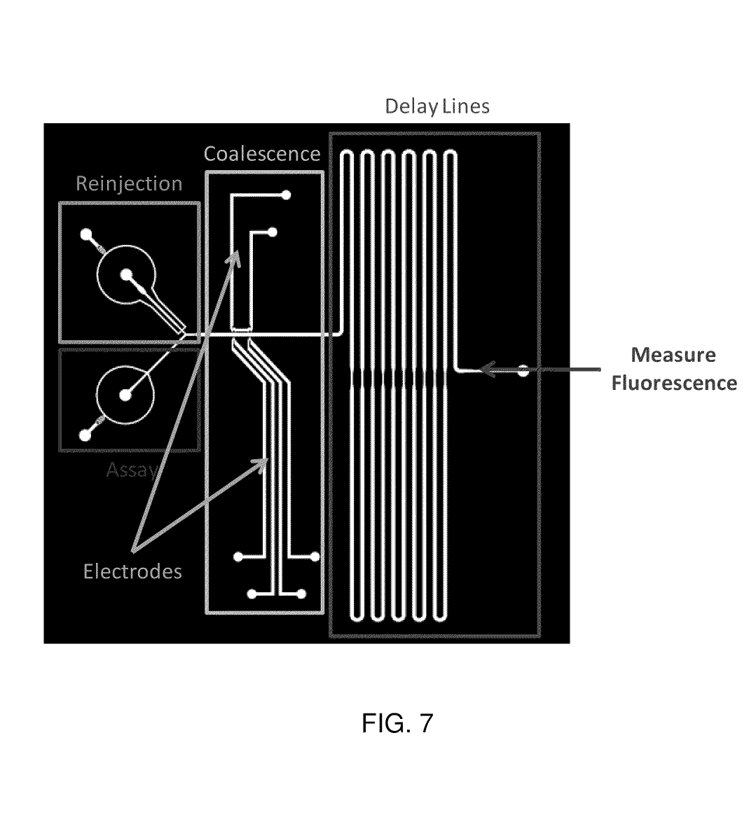

The next step was to create a droplet coalescence device with a delay line. The design for that device, which included the reinjection of the droplets with cells, the production of droplets with the assay, the coalescence of the two types of droplets, the delay lines, and the point where fluorescence is measured, is shown in FIG. 7. The channels in this device were 75 micrometers tall.

In the coalescence device, reinjected droplets and assay droplets were fed to the device in an alternating fashion in the channel leading up to the device. The assay droplets had a diameter of 225 micrometers, which was larger than the 90 micrometer reinjected droplets. When using this device, it was more desirable to observe an occasional extra large droplet rather than an extra small droplet, since having an uncoalesced assay droplet was better than coalescing two reinjected droplets with an assay droplet. Due to the parabolic velocity profile in the channels, the reinjected droplets flowed faster than the assay droplets so that by the time the droplets reached the coalescence electrodes, they were in contact with each other. See, e.g., Int. Pat. Apl. Pub. No. WO 2007/089541, published Aug. 9, 2007, incorporated herein by reference. The electrodes applied an AC potential of 1 kV at a frequency of 20 kHz which destabilized the droplet interface and caused the droplets to coalesce.

After the droplets flowed through delay lines for 30 seconds, a blue laser spot at a wavelength of 488 nm was placed in the middle of the channel to excite the fluorescent dye, resorufin. The dye emitted light and was detected by a photomultiplier tube with a filter centered at 593 nm. A custom software program written with LabView was used to analyze the detection data so that the maximum intensity of the resorufin fluorescence peaks could be recorded.

To demonstrate the functionality of this device, two strains of xylose consuming cells were cultured in droplets and the resulting xylose concentration was measured at various time points. The data from each time point were from the droplets from two syringes, and the syringes were not reused for later time points. The two strains used in the experiments were H131 and TAL1. The H131 strain consumed xylose more quickly than the TAL1 strain. To determine consumption rates, an anerobic fermentation of a 25 mL culture in a 50 mL conical tube was performed in which an HPLC was used to measure the concentrations. After 1 day of culturing, the concentration of xylose was reduced from about 5.1 g/L to about 4.5 g/L for the H131 strain, but was not significantly reduced for the TAL1 strain. After 3 days of culturing, the concentration of xylose was reduced to about 1.1 g/L for the H131 strain (from about 5.1 g/L) but only to about 3.8 g/L for the TAL1 strain (again, from about 5.1 g/L).

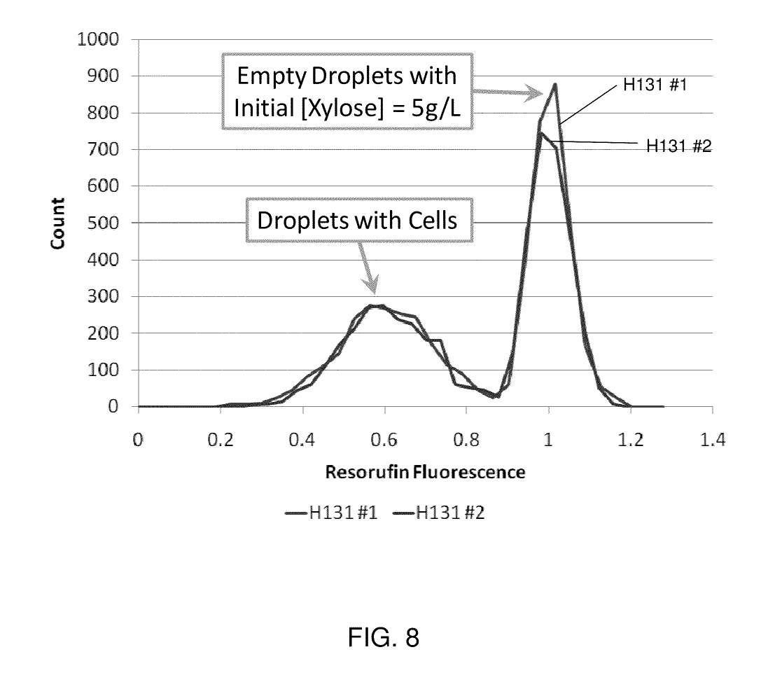

FIG. 8 shows the raw fluorescence distribution data of the H131 strain after 2 days of culturing. In this set of experiments, there was always a percentage of droplets to that did not contain any cells, and thus, an analyzed population which contains the initial xylose concentration of 5 g/L. The peak in the distribution which had the highest fluorescence value corresponded to the population of droplets that did not contain cells, while the peak in the distribution which had the low fluorescence values corresponded to the droplets which did contain cells. The existence of empty droplets allowed for the normalization of all the fluorescence data by the average fluorescence value of that peak. As can be seen in FIG. 8, the empty droplet normalized fluorescence has an average value of 1. The second normalization of the data was performed to ensure that the numbers of droplets in the two sets of data were identical.

To determine how the droplet measurement of the remaining xylose concentration compares with a measurement from a larger culture measured on an HPLC, FIG. 8 was replotted so that the abscissa was transformed to the estimated xylose concentration by using the calibration curve data in Equation 1. It was estimated that the average xylose concentration of droplets containing cells was 2.5 g/L. This value was close to the 2.7 g/L HPLC measurement of a significantly larger culture.

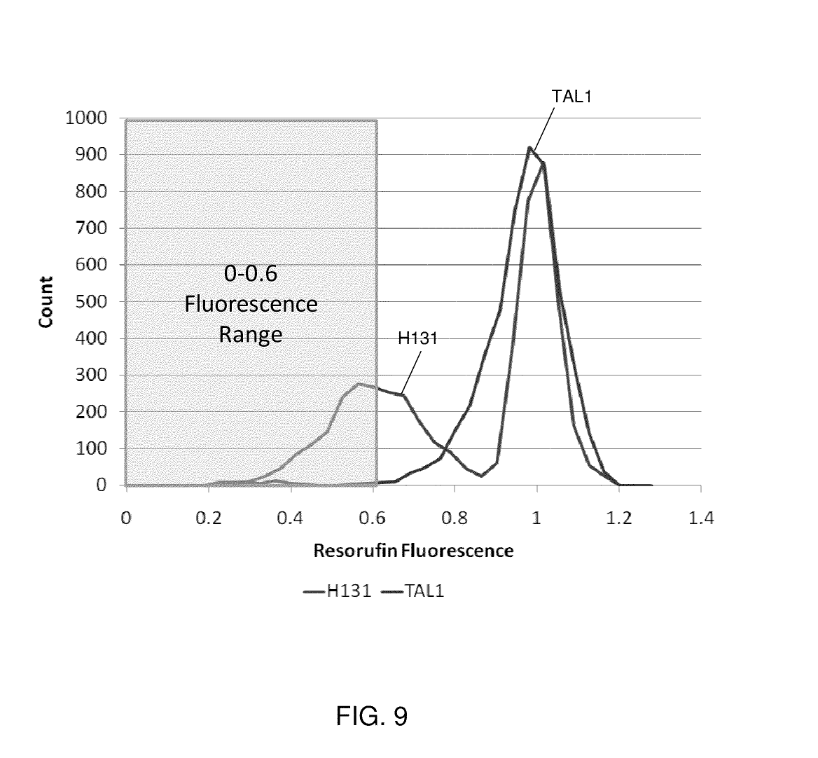

The next step was to determine if the two strains could be differentiated based on the detection data. FIG. 9 shows that qualitatively there were more droplets with low fluorescence values in the H131 strain. However, the xylose consumption of two different strains could also be quantitatively compared by calculating the percentage of droplets which have fluorescence values below a certain threshold. In FIG. 9, the threshold was set arbitrarily at 0.6.

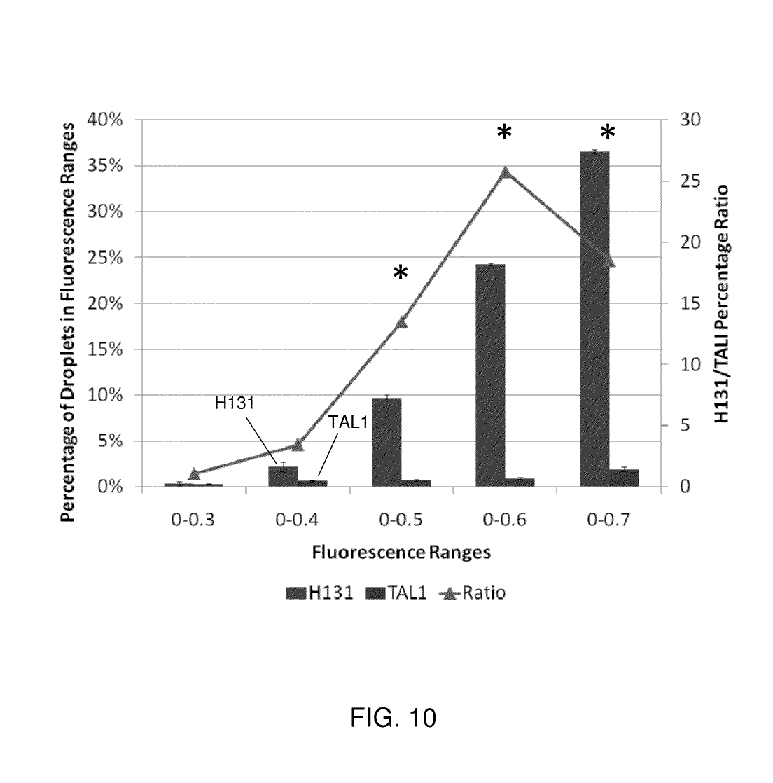

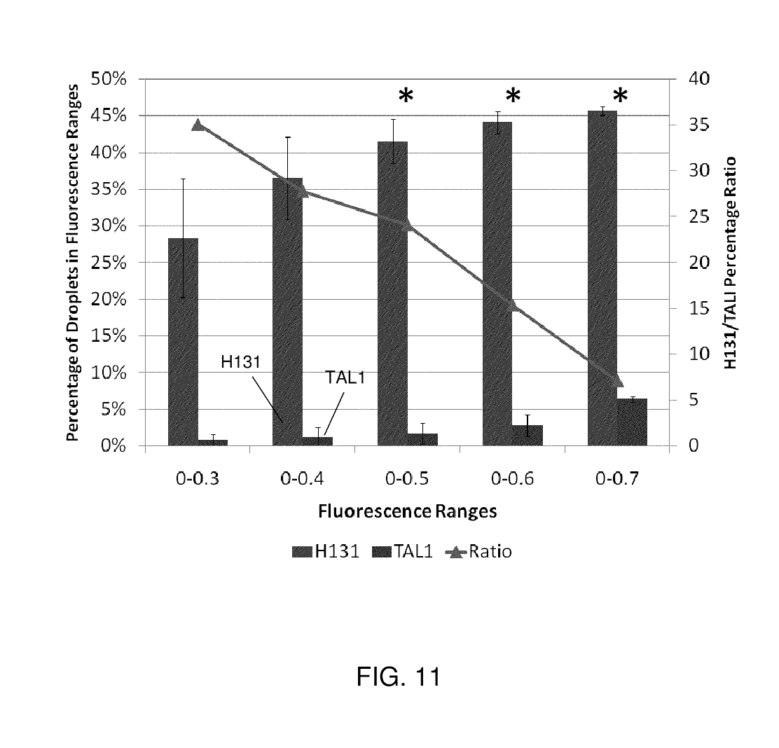

FIGS. 10 and 11 show the results of performing this analysis on data collected after 2 and 3 days of culturing, respectively. For the data after 2 and 3 days, the 0-0.5, 0-0.6, and 0-0.7 fluorescence ranges showed statistically significant differences between the two strains. Furthermore, the ratio of H131 to TAL1 droplets in those ranges was calculated to be as high as 25. This ratio was an estimation of the outgoing population if droplets were sorted based on those ranges and the incoming population was equal cell concentrations of the two strains.

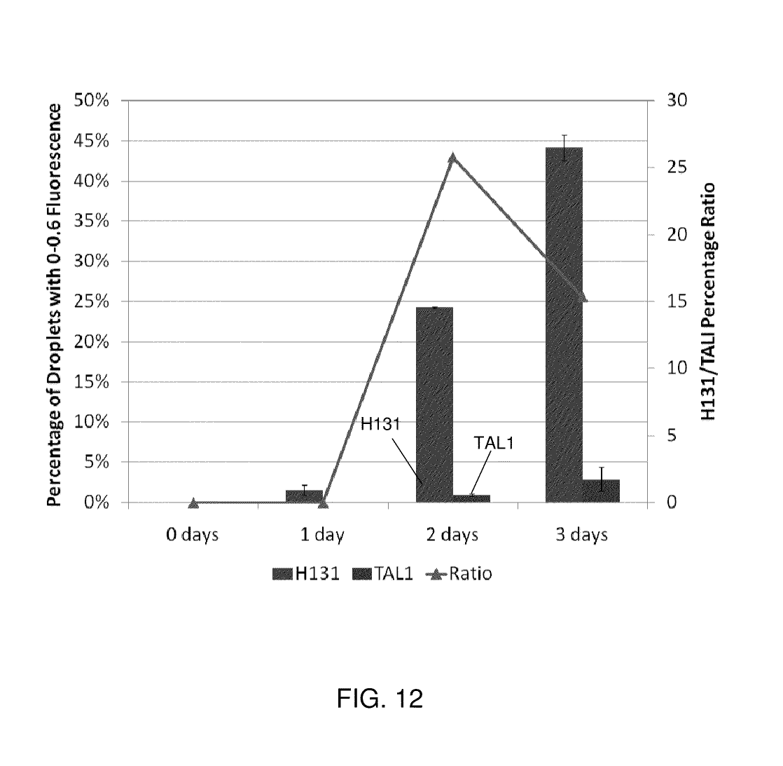

In FIG. 12, only the data for 0-0.6 fluorescence range are shown for the two strains at four different time points. The percentage of droplets increased in both strains, but the ratio decreased after 2 days because more droplets from the TAL1 strain were consuming significant amounts of xylose while most of H131 cells were already in that to bin.

An experiment was also performed in which the incoming cell population contained equal numbers of cells from the two strains to ensure that the results from that mixing the two strains together did not produce unexpected results. The 50/50 TAL1/H131 mixture had 16.6% of the droplets in the 0-0.6 bin compared with 24.3% and 0.9% for the separate H131 and TAL1 experiments, respectively. The percentage from the mixture is close to halfway in between that for the separate experiments.

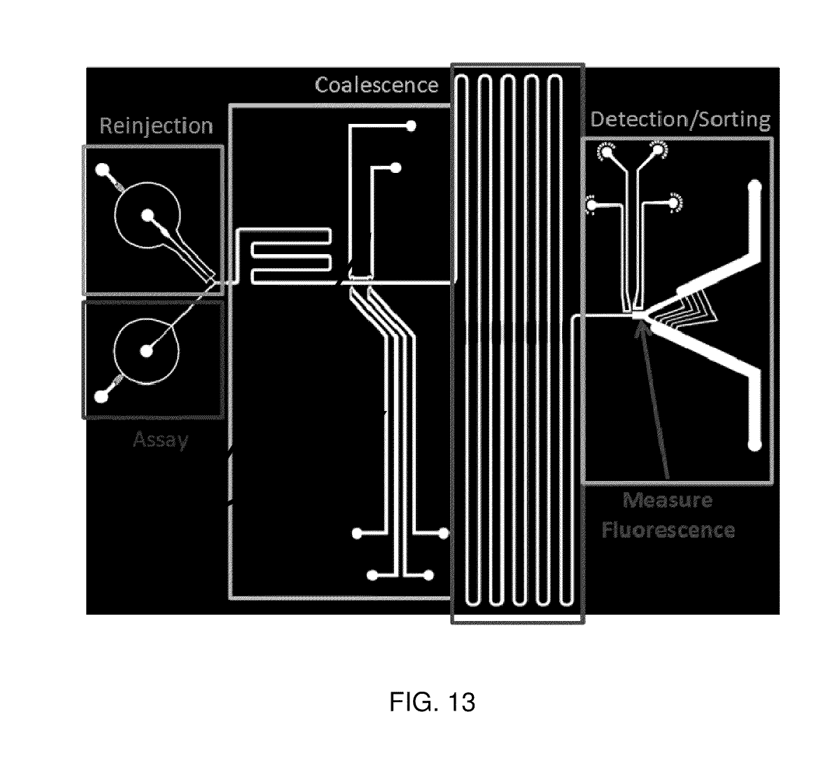

The final step for creating a complete high throughput screening device was the incorporation of a sorting device. The design for this device is shown in FIG. 13. The channels in this device were 75 micrometers tall.

The sorting portion of this device had two outlet channels. One of the channels comprised a constriction which caused it to have a higher hydrodynamic resistance. As a result, the droplets naturally flowed into the other channel. This channel became the “undesired” droplet channel. Droplets only flowed into the high hydrodynamic resistance (i.e., “desired”) droplet channel when the detection system measured a fluorescence that was within a predetermined range. The LabView software then applied a 2 kV AC pulse at 900 Hz to the electrodes. The generated AC field created an electrical potential gradient across the channel, and the droplet moved using dielectrophoresis toward the electrodes and into the “desired” droplet channel. In cases where the droplets flowing through the channels were not spaced far enough apart, the “undesired” droplets would flow into the “desired” channel. Thus, tuning the input flow rates was useful for effecting separation. The droplets in the two channels were collected using 1 mL unfiltered pipet tips.

Optimizing the flow rates of the reinjected droplets and assay droplets allowed for the control of the spacing but also increased the amount of time necessary for the pairs of droplets to come in contact with each other before coalescence. As a result, longer channels were needed in some embodiments.

The overall device was demonstrated by enriching the H131 strain with respect to the TAL1 strain. The incoming cell population contained equal numbers of both strains. Two runs of sorting were performed in which different sorting gates were used. In the first experiment, droplets below a fluorescence value of 0.7 were sorted into the “desired” channel, and in the second experiment, a gating value of fluorescence below to 0.6 was used. The H131 strain cannot grow on leucine deficient media while TAL1 can. As a result, the “desired” sorted population was grown on two types of agar plates, one which contained leucine in which both strains could grow and another which did not contain leucine which only permitted growth of TAL1. After culturing the plates, the number of colonies on each type of plate was counted. As a control, the incoming population of cells was also grown on plates. When sorting for droplets with a fluorescence less than 0.6, the population was enriched over 18 times; using a fluorescence less than 0.7, the enrichment was over 21 times. In both cases, there was a statistically significant (p<0.05) difference between the initial and sorted populations.

Anaerobic culturing was performed using H131 and TAL1 strains. After 1 hour of culturing, the cell density (OD₆₀₀) was about 0.5 for TAL1 and about 1.9 for H131. After two hours, the cell density was about 4.1 for H131 and about 1.3 for TAL1. After three hours, the cell density was about 5.7 for H131 and about 2.2 for TAL1. Based on these results, the enrichment solely from the cell growth difference between H131 and TAL1 would only be 3.2 times. Traditionally, high xylose consumption would be screened by serially subculturing an incoming cell population. This high throughput screening platform can enrich by 21 times within 2 days which shows that it is a more advantageous screening methodology.

In many cases, cell libraries contain a very low number of desired cells in the overall cell population. Hence, two test libraries with incoming desired (H131) to undesired (TAL1) cell population ratios of 1:1,000 and 1:10,000 were screened such that droplets having fluorescence in the range of 0-0.7 were sorted into the “desired” bin. A target final population ratio of 1:2.5 was defined (i.e., if 5 clones were randomly selected, one could be assured of finding an H131 cell). This target was achieved after only one round of screening with the 1:1,000 library, and after two rounds with the 1:10,000 library. The sequence for one round of screening was a preculture of the incoming cells, a shake flask culture which was grown into early exponential phase, droplet encapsulation of cells, and selection of droplets with low xylose concentrations. One round of screening enriched the 1:1,000 library by 420 times, and two rounds enriched the 1:10,000 library by 42,600 times.

### Example 2

In this example, the nature of the genetic modification(s) underlying the superior xylose uptake performance that some strains acquired as a result of the evolution was investigated. The H131-A31 strain, used in this example, was similar to H131 with the exception of one important difference: instead of the XYL1 and XYL2 genes, H131-A31 contained the Piromyces sp. E2 XYLA gene encoding a xylose isomerase enzyme to convert D-xylose to D-xylulose. This strain initially exhibited negligible growth and xylose consumption rates. After evolving it over a period of several months through growth and serial subculturing, strain H131E-A31 was obtained that was characterized by high growth (μ˜0.2 hr⁻¹) and high xylose consumption rates (14 g/L in 2 days).

To identify the genetic elements responsible for the improved performance of the H131E-A31 strain, a genomic library of this strain was constructed and transformed into H131-A31. H131-A31 is similar to H131 except its genotype is MAT_a, leu2, ura3, arg4, ade1::ADE1-GPD-PsTAL1, trp1:TRP1-GPDP-ScRKI1-ScRPE1, his2::HIS2-GPDP-ScTKL1 with pRS426-GPDP-XYLACYCT-GPDP-XYL3-CYCT. XYLA is the xylose isomerase gene from Piromyces sp. E2. H131EA31 is the evolved version of H131-A31.

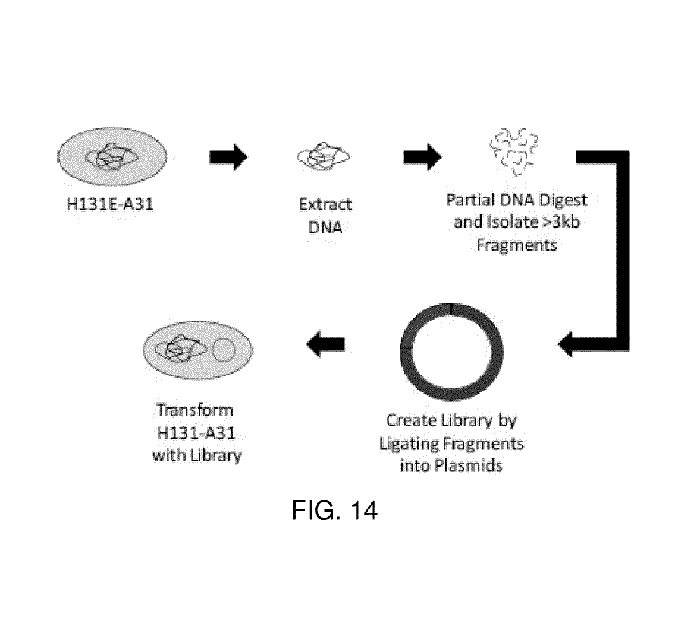

The genomic DNA library construction is summarized in FIG. 14. The genetic background in which the library was transformed was the H131-A31 strain. The library was constructed by performing a genomic DNA prep of H131E-A31 using the Wizard Genomic DNA purification kit (Promega) and partially digesting the DNA with Sau3AI (New England Biolabs). Fragments larger than 3 kb were selected on an agarose gel and the DNA was gel purified and purified again with ethanol precipitation. pRS415 was used as the backbone and digested with SalI. Both the insertion fragments and the backbone were incubated with the Klenow fragment of DNA polymerase I and the appropriate dNTPs to reduce the overhang length from 4 to 2 basepairs to decrease the frequency of self-ligation. The backbone was also dephosphorylated to prevent self-ligation. After ligating the fragments and the backbone together with T4 ligase, the resulting plasmid was transformed into ElectroMAX™ DH5α-E (Invitrogen) and plated on ampicillinresistant agar petri dishes. This DH5α library contained 10⁶ colonies. The plasmid was miniprepped and transformed into the unevolved H131-A31 strain using the Frozen-EZ Yeast Transformation II™ kit (Zymo Research). The resulting yeast library to contained 5×10⁵ colonies.

The library was constructed such that each insertion had a high probability of containing at least one open reading frame. Assuming that a single mutation, rather than a combination of multiple mutations, was sufficient to yield cells with improved xylose assimilation rate, the system was capable of isolating a mutant harboring a single genomic fragment by screening the cell population transformed with the library. The library contained 5×10⁵ clones; after only one round of screening, mutant W2 was isolated as the one having the highest xylose consumption rate. Cumulative xylose consumption over the course of 4 days was measured for strain H131-A31 transformed with an empty plasmid (control), mutant W2, and strain H131-A31 with the plasmid isolated from mutant W2 (retransformed W2). The control consumed about 0 g/L of xylose over the course of four days. The retransformed W2 consumed about 0.2 g/L after day 1, about 1 g/L after day 2, about 1.8 g/L after day 3, and about 2.6 g/L after day 4. The control consumed about 0 g/L of xylose over the course of four days. The mutant W2 consumed about 0.8 g/L after day 1, about 2.2 g/L after day 2, about 3.7 g/L after day 3, and about 4.7 g/L after day 4. Biological replicates were used in these measurements. The difference in xylose consumption for the retransformed W2 strain and the mutant W2 after four days of culturing suggested that a background mutation also occurred in the W2 mutant. However, both consumed more xylose than the control which confirmed there was a mutation on the plasmid which provided a benefit over the control.

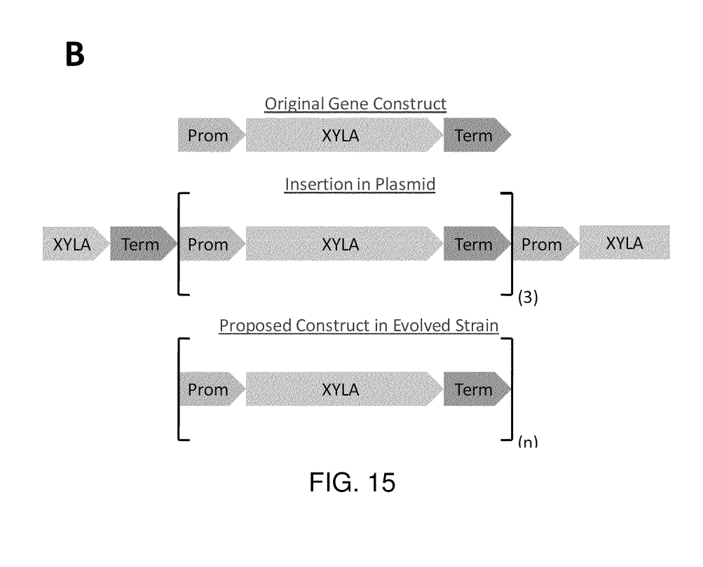

Sequencing and restriction enzyme digest analysis determined that the plasmid isolated from the W2 strain contained 3 full copies of the XYLA gene construct flanked by truncated XYLA sequences (FIG. 15). The exact number of XYLA copies in H131E-A31 is unknown but it probably contained at least 5 copies which is an increase from the original single copy in H131-A31. H131-A31E should contain the three full XYLA genes seen in the W2 plasmid since the gene duplication most likely occurred during the several month evolution as opposed to the several day growth of the library. Since the truncation sites for the partial XYLA genes matched the restriction enzyme sites for Sau3AI (the enzyme used to partially digest the DNA from the H131E-A3) larger pieces of the XYLA gene likely existed in that strain. Since duplication of full gene constructs has already been observed, these larger pieces were most likely full constructs which increases the total number of copies to five.

The xylose isomerase gene catalyzed the reaction which initiated xylose assimilation by the cell. Additional copies of XYLA would allow for increased xylose uptake and cell growth. As the growth of the original H131-A31 strain was relatively slow on xylose, the selection pressure of xylose being the sole carbon source in the medium led to the enrichment of cells harboring increased copies of XYLA because of the growth advantage that such cells would enjoy in a xylose medium. These multiple copies of XYLA were linked so they were created through the naturally occurring process of tandem gene duplication where recombination occurs between two sites which have non-identical locations. Typically, gene duplication occurs at approximately the same rate as point mutations. The pRS426 plasmid from the H131-A31 strain contained not only the XYLA gene flanked by a promoter and terminator but also P. stipitis XYL3 with identical flanking regions. These homologous flanking regions would allow tandem gene duplication to occur during plasmid replication. Furthermore, since the pRS426 plasmid is also a multicopy plasmid, its replication occurs more frequently than mitosis which would increase the probability of a duplication event. Quantitative PCR was performed to determine the number of copies of XYLA in the H131-A31 and H131E-A31 strains. There were 1.3±0.3 copies in H131-A31 and 47.9±9.0 copies in H131E-A31 normalized to the copies of the PGK gene which also confirms the increase number of copies of XYLA after evolution.

DNA sequencing also identified a serine to tyrosine point mutation (Ser19Tyr or S19Y) in the xylose isomerase enzyme. The protein crystal structure for xylose isomerase from Thermotoga Neapolitana (having 52% amino acid sequence identity to xylose isomerase from Piromyces that was used in the construction of strain H131-A31) has been solved. This occurs on the outer shell of the protein away from the active site so it is not believed to have affected the activity of the enzyme. Furthermore, when a version of H131-A31 was created with the S19Y mutation, this strain did not exhibit improved growth when compared to the strain with the unmutated XYLA. Thus, the gene duplication of the XYLA gene construct may have been the main cause for the improved W2 strain from the library.

### Example 3

This example describes a method for identifying high ethanol producing strains of S. cerevisiae by identifying high glucose consuming strains. Glucose consumption and ethanol production are correlated. For example, ATCC 24858 is capable of reducing the concentration of glucose from about 4.6 g/L to about 4.2 g/L after 3 hours, about 3.3 g/L after 5 hours, about 2 g/L after 7 hours, and about 0.5 g/L after 9 hours. At the same time, the ethanol concentration rises to about 0.06% after 5 hours, about 0.18% after 7 hours, and about 0.25% after 9 hours. ATCC 24858 consumes more glucose and also produces more ethanol while the adh1 knockout strain (adh1 KO) consumes very little glucose and produces negligible amounts of ethanol. PDC1-GFP is a medium producing and consuming strain. Thus, the assumption of using an indirect measurement of selection is valid. In this example, Amplex UltraRed is used to detect glucose oxidase to perform the screen.

The three strains used in this example were ATCC 24858, BY4741 PDC1-GFP (PDC1-GFP), and BY4741 Δadh1 (adh1 KO). ATCC 24858 is an industrial polyploid S. cerevisiae strain (Ness, Lavallee, Dubourdieu, Aigle, & Dulau, 1993). BY4741 PDC1-GFP is BY4741 with a green fluorescent protein (GFP) linked to the pyruvate decarboxylase (PDC1) gene (Huh et al., 2003). BY4741 Δadh1 is the BY4741 strain with a deletion of the main alcohol dehydrogenase (adh1) gene (Winzeler et al., 1999).

The yeast fermentations in this example were performed in 250 mL Erlenmeyer flasks at 30° C. using an orbital shaker rotation speed of 225 rpm. Microaerobic fermentations were performed by bubbling nitrogen through the shake flask contents and sealing with a rubber stopper with a needle. The culturing medium contained 6.7 g/L yeast nitrogen base without amino acids (Difco), complete synthetic medium (MP Biomedicals) with the appropriate amino acid dropouts to maintain the plasmids, and 5 g/L glucose.

The culture medium in the droplets contained 1× yeast nitrogen base without amino acids (Difco), complete synthetic medium (MP Biomedicals) with the appropriate amino acid dropouts to maintain the plasmids, and 5 g/L glucose. Microaerobic culturing of droplets occurred at 30° C. in a capped 1 mL syringe.

The microfluidic droplet screening system described in Example 1 was used to culture yeast cells in droplets, measure the remaining amount of glucose using the Amplex UltraRed/glucose oxidase enzyme system, and select for high glucose to consuming strains.

For the quantification of glucose and ethanol concentrations from shake flasks, cell-free culture supernatants were filtered through 0.2-μm-pore-size polytetrafluoroethylene membrane syringe filters (VWR International). These samples were analyzed in a high-performance liquid chromatography (HPLC) system with a Waters 2690 Separations module connected with a Waters 410 refractive index detector (Waters). The samples were separated on a BioRad Aminex HPX-87H ion exclusion column for organic acid analysis with 14 mM sulfuric acid as the mobile phase at a flow rate of 0.7 mL/min. For cell density determinations, the optical densities of cultures were measured at 600 nm using an Ultrospec 2100 pro UV/visible spectrophotometer (Amersham Biosciences).

The glucose consumption of a strain cultured in a droplet was compared to that in a shake flask. The fluorescence distributions were converted to an estimated glucose distribution by using a calibration curve collected in previous experiments. The data in these distributions were collected from biological replicate experiments. The distribution of the droplets containing cells was broader than in the xylose experiments. Since the glucose consumption by these strains was much faster than the xylose consumption of the engineered xylose strains, it is possible that the higher sugar consumption rate was responsible for these wider distributions.

The average glucose consumption of the ATCC 24858 strain after 3 hours was approximately 4 g/L which was similar to the 4.3 g/L shake flask glucose concentration at the same time point. A similar analysis was performed on the BY4741 PDC-1 GFP strain. After culturing for 7 hours, the average glucose concentration in the droplet was 3 g/L which approximated the shake flask concentration of 3.4 g/L.

By culturing the individual strains and analyzing the fluorescence detection data at different time points, one can determine if the screening system can differentiate the strains. The percentage of droplets with fluorescence values less than 0.6 from the three yeast strains were analyzed over the period of several hours. For ATCC 24858, the percentage of droplets with fluorescence in this range was about 3% after 2 hours, about 10% after 3 hours, and about 14% after 4 hours. For PDC-1 GFP, about 1% of the droplets fell within 0.0 to 0.6 fluorescence range after 3 and 4 hours, about 8% after 5 to hours, about 14% after 6 hours, and about 18.5% after 7 hours. For adh1KO, about 1% of the droplets fell within 0.0 to 0.6 fluorescence range after 5 hours and about 2.5% fell within that range after 7 hours. The values were calculated for each time point and showed an identical glucose consumption trend compared to the measurements from the shake flask experiments. The trend shows that the strains in order of fastest to slowest consumption are ATCC 24858, BY4741 PDC1-GFP, and BY4741 Δadh1.

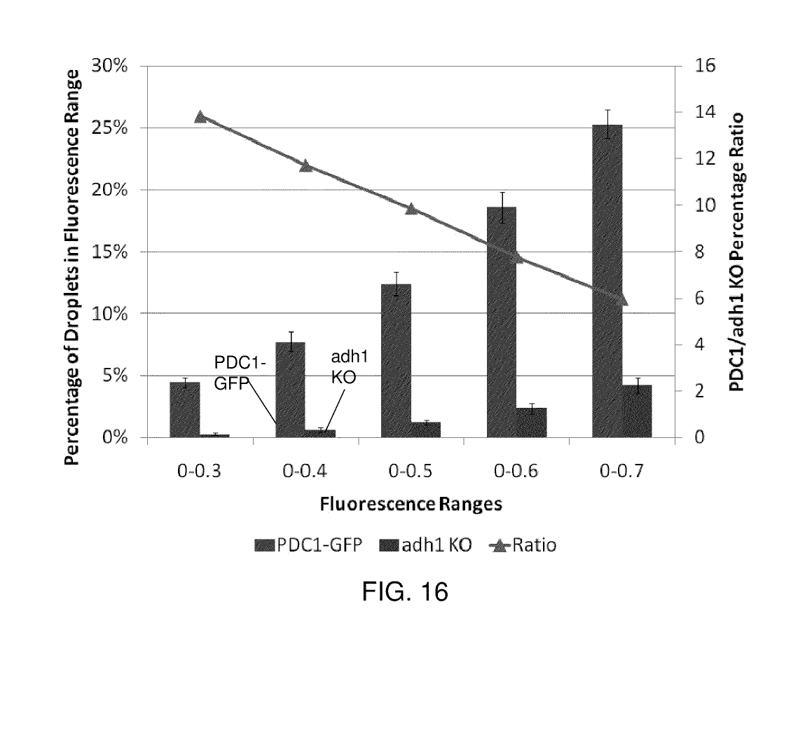

The BY4741 PDC1-GFP strain was enriched from an equal proportion mixture of it and the BY4741 Δadh1 strain. Before performing the actual enrichment experiment, each strain was grown separately in droplets for 7 hours with biological replicates, and the resulting fluorescence data for the percentage of droplets in different fluorescence ranges was analyzed (FIG. 16). There was a statistical difference between the two strains for the data in the 0-0.5, 0-0.6, and 0-0.7 ranges. The percentage ratio for a bin range is the ideal enrichment of PDC1-GFP when there is no sorting error. The highest ratios for the statistically significant ranges were 0-0.5 and 0-0.6. Thus, these ranges were chosen as the sorting thresholds for the enrichment experiment. The cells for the enrichment study were cultured for 7 hours. The enrichment due to growth was almost 19× because the PDC1-GFP strain grew faster than the Δadh1. Sorting improved this enrichment by to 42× when using the 0.0 to 0.5 fluorescence band, In addition, sorting improved this enrichment by additional 3× when using the 0.0 to 0.6 fluorescence band, for a total enrichment of 54×.

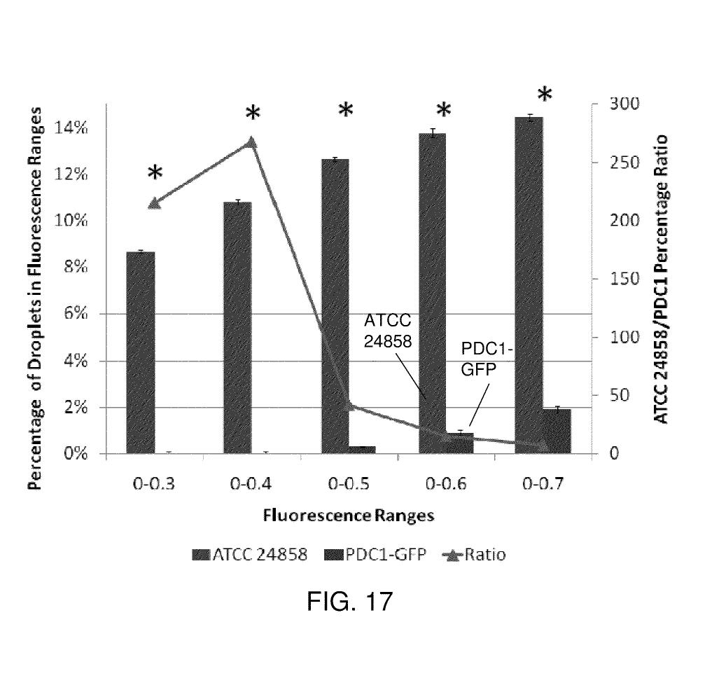

A second enrichment experiment for a ATCC 24858 and BY4741 PDC1-GFP mixture was performed. Analysis of the detection data when culturing each strain separately showed that the less than 0.3 through 0.7 fluorescent ranges all showed a statistically significant difference between the two strains (FIG. 17). Furthermore, the 0-0.4 bin range showed the highest amount of ideal enrichment and was chosen as the range used in the actual sorting experiment which enriched for the ATCC 24858 strain by over 6×. It was also observed that there was no growth contribution to the enrichment, as evidenced by the substantial lack of enrichment over 4 hours when no sorting was performed.

### Example 4

In this example, Escherichia Coli strains were screened to isolate high xylose consuming strains using the high-throughput screening system. It may be desirable, in to some instances, to identify strains of E. coli that produce high amount of ethanol. However, ethanol production can be difficult to measure in some systems. As an alternative, xylose consumption can be measured. Previous E. coli library work has shown that there is a relatively strong correlation between xylose consumption and ethanol production, which can be expressed as:

Xylose Consumption (g/L)=(2.2408*Xylose Concentration (g/L))+0.0664 [2]

The correlation in Equation 2 resulted in an R² value of 0.9244.

The parental strain used in this example was XZ030, which was provided by Verenium Corporation. This strain is similar to the KO11 strain (Yomano et al., 1998). Errorprone PCR was used to create the rpoA and rpoD libraries. The rpoA and rpoD libraries were constructed using plasmids pCL1920 and pHACM, respectively. The mutations for the rpoA library were targeted to the C-terminal domain.

The E. coli microaerobic fermentations used in this example were performed with 5.5 mL of medium in KingFisher 24-well deep well plates (Thermo Fisher) covered with aluminum foil at 35° C. using an orbital shaker rotation speed of 225 rpm. All culture medium contained the appropriate antibiotics to maintain the plasmids, and the desired concentration of xylose. The rich culturing medium contained overlimed hydrolysate from sugar cane at pH 6.5 with 5% corn steep liquor and a total of 140 g/L xylose. The minimal medium was AM1 with 30 g/L ethanol and either 10 or 20 g/L xylose (Martinez et al., 2007). Microaerobic culturing of cells in droplets occurred at 37° C. in a capped 1 mL syringe.

The rpoA library was pre-screened before microfluidic droplet screening by growing cells in overlimed hydrolysate from sugar cane at pH 6.5 with 5% corn steep liquor, 100 g/L xylose, and 80 g/L ethanol for 4 hours. The rpoD library was prescreened in a similar manner except 50 g/L ethanol was used and the cells were exposed to that environment for 6 hours.

The microfluidic droplet screening system was used to culture the bacterial cells in droplets, measure the remaining amount of xylose using Amplex UltraRed to indicate pyranose, and select for high xylose consuming strains. For the quantification of xylose concentrations from shake flasks, cell-free culture supernatants were filtered through 0.2-μm-pore-size polytetrafluoroethylene membrane syringe filters (VWR International). These samples were analyzed in a high-performance liquid chromatography (HPLC) system with a Waters 2690 Separations module connected with a Waters 410 refractive index detector (Waters). The samples were separated on a BioRad Aminex HPX-87H ion exclusion column for organic acid analysis with 14 mM sulfuric acid as the mobile phase at a flow rate of 0.7 mL/min. For cell density determinations, the optical densities of cultures were measured at 600 nm using an Ultrospec 2100 pro UV/visible spectrophotometer (Amersham Biosciences).

Since single E. coli cells can be difficult to visualize on an optical microscope, an E. coli strain which expressed a superfolder green fluorescent protein was used to determine the optimal incoming cell density to ensure that there is one cell in every 2-3 droplets (Pedelacq, Cabantous, Tran, Terwilliger, & Waldo, 2006). The high emitted fluorescence of this strain allowed for low magnification imaging of the cells in droplets which was necessary because the droplets were 75 μm in diameter while the cells were only 1 μm wide and several microns long. By visualizing the cells and droplets simultaneously on a microscope, the optimal cell density was determined to be OD₆₀₀=0.003.

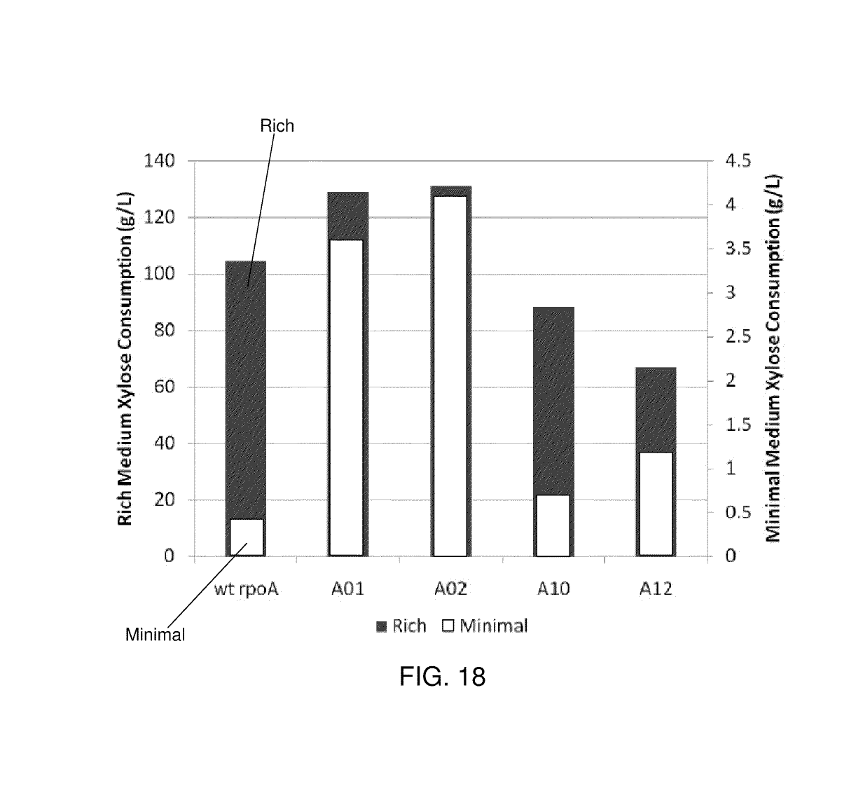

The bulk cultures were performed in a rich medium with 140 g/L xylose. The Amplex UltraRed/pyranose oxidase enzyme system was not compatible with a rich medium since they contain compounds which will react with the horseradish peroxidase enzyme to produce a high background signal. Furthermore, the medium was also opaque which can cause problems during fluorescence detection. Thus, a minimal medium was used to culture the cells in the screening system. The AM1 minimal medium had been developed to culture ethanologenic bacterial strains and was used to culture cells in droplets (Martinez et al., 2007). Furthermore, 140 g/L xylose was challenging to measure in the assay system. At high xylose concentrations, the fluorescence intensity decreases. When the pyranose oxidize enzyme concentration was decreased to a level at which the fluorescence increased or remained constant at high xylose concentrations, the peak to background fluorescence ratio became significantly lower than the ideal ratio of 10. Thus, it was determined that an initial 20 g/L xylose concentration was the highest concentration that would be used with the assay system. Since 140 g/L xylose also stressed the cells, 30 g/L ethanol was added to the culturing medium as a different stress to the cells. To ensure that high xylose consumers in the rich medium with 140 g/L xylose perform similarly in minimal medium with 20 g/L xylose and 30 g/L ethanol, five strains from an rpoA library were cultured in both media for 72 hours and the xylose consumption was measured (FIG. 18). There was good correlation between the two types of media.

The rpoA library contained 5×10⁵ colonies. To reduce the size of the library, it was prescreened before microfluidic droplet screening by growing cells in overlimed hydrolysate from sugar cane at pH 6.5 with 5% corn steep liquor, 100 g/L xylose, and 80 g/L ethanol for 4 hours. The resulting library contained 4.2×10⁴ colonies after the stress. These colonies were screened in the microfluidic droplet system with a 60.5 hour culturing time using the AM1 minimal medium with 10 g/L xylose and 30 g/L ethanol, which was added as a stress for the cells, and 14.2% of the droplets were selected. Sixty of the selected clones were cultured for 72 hours in 24-well deep well plates using the overlimed hydrolysate/corn steep liquor rich medium mixture which contained 140 g/L xylose.

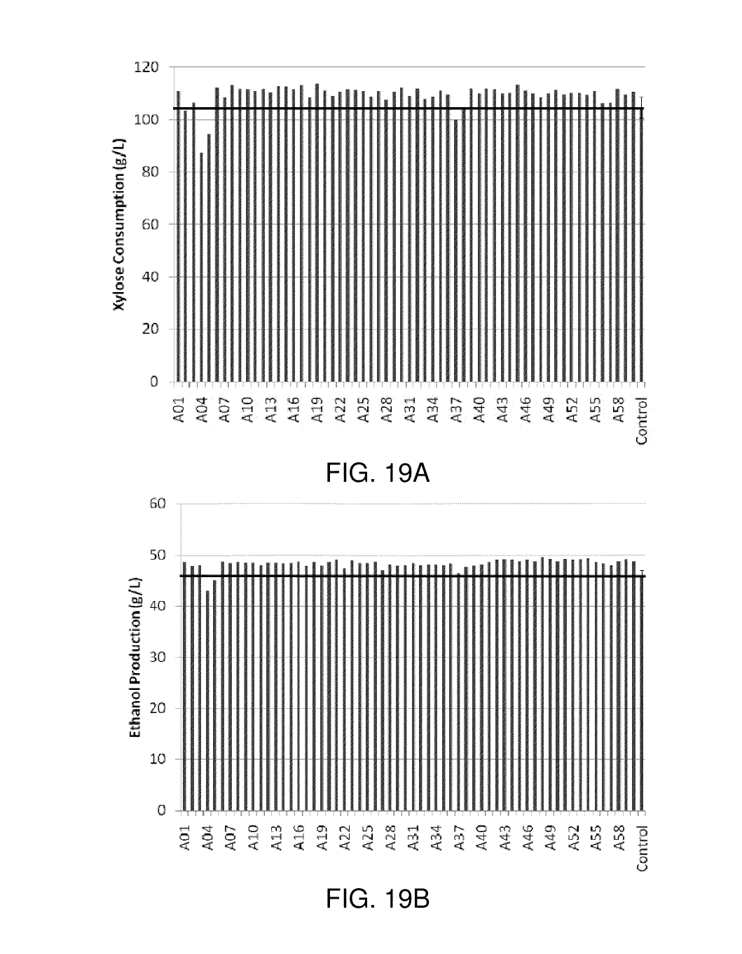

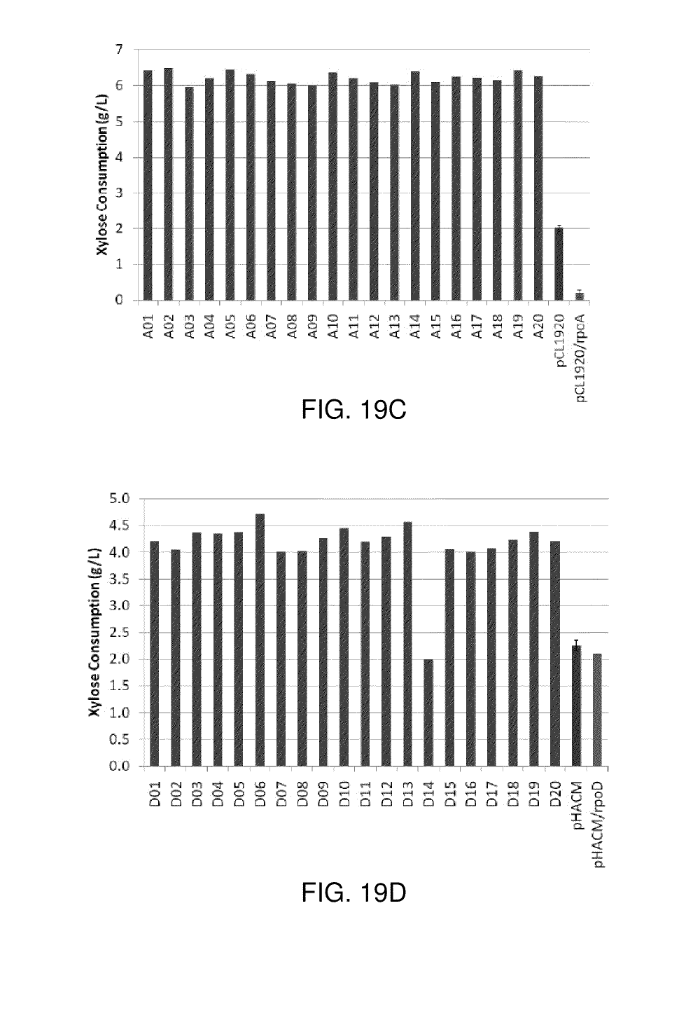

The control, which contained a plasmid with the wild type rpoA, consumed 104.3 g/L xylose and produced 46 g/L ethanol on average. The best mutant had a 113.6 g/L xylose consumption and a 48.9 g/L ethanol production, which were 8.9% and 6.3% higher than the control average, respectively (FIGS. 19A-19B). The horizontal black line in the graphs is the average concentration of the control strain. Furthermore, 93.7% of the 60 mutants had higher xylose consumption and ethanol production than the control.

Twenty of the selected rpoA mutants where also grown microaerobically in AM1 minimal medium with 20 g/L xylose and 30 g/L ethanol for 72 hours. In this experiment, the controls contained the plasmid with no insertion (pCL1920) or the wild type rpoA (pCL1920/rpoA), and all of the strains exhibited higher xylose consumption than the controls.

The xylose consumption by the selected mutant population was 6.2±0.3 g/L while it was 2.0±0.1 g/L for the strain with the blank plasmid and 0.2±0.1 g/L for the strain with the plasmid containing the wild type rpoA (FIG. 19C). Since significant amounts of ethanol were a component in the medium, there was no significant difference in the ethanol concentrations for the different strains.

The rpoD library was prescreened in a similar manner as the rpoA library except 50 g/L ethanol was used and the cells were exposed to that environment for 6 hours. After performing droplet screening in the same manner as the rpoA library and selecting 2.6% of the droplets, twenty strains were cultured in deep well plates for 72 hours. The best strain consumed 109.5 g/L xylose and produced 47.8 g/L ethanol while a strain an extra wild type rpoD on a plasmid consumed 109.7 g/L xylose and produced 47.3 g/L ethanol. In this case, the best strain was identical to the control.

However, when twenty of the strains were grown for 72 hours in AM1 minimal medium with 20 g/L xylose and 30 g/L ethanol, nineteen of these strains had considerably higher xylose consumption than the controls (FIG. 19D). The two control strains contained a plasmid with no insertion (pHACM) or with the wild type rpoD (pHACM/rpoD). These results were similar to those observed in the rpoA experiment. Excluding strain D14, the xylose consumption of screened strain population was 4.2±0.5 g/L while the controls were 2.3±0.1 g/L and 2.1±0.0 g/L for the blank plasmid and wild type rpoA strains, respectively. Since significant amounts of ethanol were a component in the medium, there was no significant difference in the ethanol concentrations for the different strains.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of shall be closed or semi-closed transitional phrases, respectively, as set forth in the United States Patent Office Manual of Patent Examining Procedures, Section 2111.03.

What is claimed is:

1. A method of producing an enriched population of cells, comprising:
providing a first population of droplets contained within a microfluidic device, at least some of which droplets encapsulate one or more cells, at least some of which droplets include a first cell type and at least some of which droplets include a second cell type;
for at least some of the droplets, determining the ability of one or more cells within the respective droplets to react with a sugar, wherein the first cell type is able to metabolize the sugar to a greater degree than the second cell type; and
based on the determination, producing an enriched population of droplets of cells of the first cell type relative to the second cell type.

2. The method of claim 1, wherein the sugar is xylose.

3. The method of claim 1, wherein the sugar is glucose.

4. The method of claim 1, wherein the amount of enrichment of the first cell type relative to the second cell type is a factor of at least about 10.

5. The method of claim 1, wherein the amount of enrichment of the first cell type relative to the second cell type is a factor of at least about 100.

6. The method of claim 1, wherein the amount of enrichment of the first cell type relative to the second cell type is a factor of at least about 1000.

7. The method of claim 1, wherein the act of producing an enriched population of droplets of cells of the first cell type relative to the second cell type comprises directing at least some of the cells of the first cell type to a first location within the microfluidic device and directing at least some of the cells of the second cell type to a second location within the microfluidic device

8. The method of claim 7, wherein the cells are directed to the first location and/or the second location by applying an electric field to the cells.

9. The method of claim 1, further comprising sequencing DNA of the first cell type.

10. The method of claim 1, wherein the first cell type and the second cell type arise from the same species.

11. The method of claim 1, wherein the first cell type and the second cell type arise from different species.

12. The method of claim 1. wherein the act of determining the ability of the one or more cells within the droplet to react with a sugar comprises determining the ability of the one or more cells to react with the sugar using fluorescence.

13. The method of claim 1, further comprising culturing at least a portion of the enriched population of droplets of cells.

14. A method of producing an enriched population of cells, comprising:
providing a population of droplets contained within a microfluidic device, at least some of which droplets encapsulate one or more cells, at least some of which droplets of the population of droplets include a first cell type and at least some of which droplets include a second cell type;
for at least some of the droplets, determining the ability of one or more cells within the droplet to react with an agent, wherein the first cell type reacts with the agent to a greater degree than does the second cell type; and
based on the determination, producing an enriched population of droplets of cells of the first cell type relative to the second cell type.

15. The method of claim 14, wherein the act of producing an enriched population of droplets of cells of the first cell type relative to the second cell type comprises directing at least some of the cells of the first cell type to a first location within the microfluidic device and directing at least some of the cells of the second cell type to a second location within the microfluidic device

16. The method of claim 15, wherein the cells are directed to the first location and/or the second location by applying an electric field to the cells.

17. The method of claim 14, wherein the agent is a sugar that is metabolized by at least the first cell type.

18. The method of claim 14, wherein the agent is xylose.

19. The method of claim 14, wherein the agent is glucose.

20-36. (canceled)

37. A method, comprising:
providing a population of droplets contained within a microfluidic device, at least some of which droplets encapsulate one or more cells and at least some of which droplets contain a sugar;
exposing at least some of the droplets to an enzyme able to react with the sugar; and
determining an extent of reaction of the enzyme with the sugar.

38-65. (canceled)
